SEQUENCE LISTING (12 sequences)

SEQ ID NO: 1 - DNA, 25 bp, Artificial sequence
  Single strand DNA oligonucleotide
tgttcaaccaattatcagcaaactc

SEQ ID NO: 2 - DNA, 24 bp, Artificial sequence
  Single strand DNA oligonucleotide
ttctgctgtatcctcttcaccagt

SEQ ID NO: 3 - DNA, 22 bp, Artificial sequence
  Single strand DNA oligonucleotide
agaaccaggaaacggaaacaga

SEQ ID NO: 4 - DNA, 20 bp, Artificial sequence
  Single strand DNA oligonucleotide
tctccttcatgcgctgcttt

SEQ ID NO: 5 - DNA, 21 bp, Artificial sequence
  Single strand DNA oligonucleotide
ctgagtccgaatcaggtgcag

SEQ ID NO: 6 - DNA, 21 bp, Artificial sequence
  Single strand DNA oligonucleotide
atccatggggagatgttctgg

SEQ ID NO: 7 - DNA, 20 bp, Artificial sequence
  Single strand DNA oligonucleotide
agccctagtagaggttggca

SEQ ID NO: 8 - DNA, 19 bp, Artificial sequence
  Single strand DNA oligonucleotide
aggagccatgtaggaaggc

SEQ ID NO: 9 - DNA, 20 bp, Artificial sequence
  Single strand DNA oligonucleotide
ctgtgtgctggagttacgct

SEQ ID NO: 10 - DNA, 20 bp, Artificial sequence
  Single strand DNA oligonucleotide
tactcctgcaaatcctgagc

SEQ ID NO: 11 - DNA, 20 bp, Artificial sequence
  Single strand DNA oligonucleotide
caacggatttggtcgtattg

SEQ ID NO: 12 - DNA, 20 bp, Artificial sequence
  Single strand DNA oligonucleotide
gatgacaagcttcccgttct

(19) United States
(12) Patent Application Publication
(10) Pub. No.: US 2019/0046497 A1
(43) Pub. Date: Feb. 14, 2019

(54) METHODS OF MODULATING PROTEIN EXOCYTOSIS AND USES OF SAME IN THERAPY

(71) Applicant: Yeda Research and Development Co. Ltd., Rehovot (IL)

(72) Inventors: Yifat MERBL, Rehovot (IL); Ron BENYAIR, Rehovot (IL)

(73) Assignee: Yeda Research and Development Co., Ltd., Rehovot (IL)

(21) Appl. No.: 16/077,511

(22) PCT Filed: Feb. 14, 2017
(86) PCT No.: PCT/IL2017/050189

§ 371 Date: Aug. 13, 2018

Related U.S. Application Data
(60) Provisional application No. 62/295,086, filed on Feb. 14, 2016.
(60) Provisional application No. 62/358,018, filed on Jul. 3, 2016.

(51) Int. Cl. A61K 31/35; A61P 35/00; A61K 31/472; A61K 31/365; C12Q 1/6883; C12Q 1/6886; A61P 37/06

(52) U.S. Cl. CPC A61K 31/35; A61P 35/00; A61K 31/472; C12Q 2600/106; C12Q 1/6883; C12Q 1/6886; A61P 37/06; A61K 31/365

(57) ABSTRACT

A method of modulating protein exocytosis is provided. The method comprising contacting a cell with an agent that modulates the ubiquitin pathway in the Golgi, thereby modulating protein secretion.

### FIELD AND BACKGROUND OF THE INVENTION

The present invention, in some embodiments thereof, relates to methods of modulating protein exocytosis and uses of same in therapy.

Proteins in the mammalian cell are synthesized by ribosomes, either in the cytosol or bound to the ER membrane. Upon completion of synthesis, and in some cases even before synthesis is concluded, these proteins begin to undergo stringent quality control processes that ensure their proper folding (1, 2). In the ER, proteins will undergo various modifications such as N-linked glycosylation and disulfide bond formation, and will be probed for their folding state. Quality Control (QC) processes have been extensively studied, especially in the context of ER quality control, where proteins can be bound by chaperones, probed by folding sensors and, if need be, retrotranslocated to the cytosol for degradation by the UPS.

Polyubiquitylation is a vital step in the retrotranslocation (3) as well as the degradation of ER associated degradation (ERAD) substrates. Coupling the QC machinery in the ER with ubiquitin E3 ligase enzymes in the ER membrane allows strict control over the processing and degradation of proteins in the secretory pathway by counterbalancing the opposing actions of ubiquitin E3 ligases and deubiquitylating enzymes (DUBs) (4). Following QC in the ER, a secretory protein that has been deemed properly folded will exit the ER through COP-II coated ER exit sites, bound for the Golgi apparatus.

Once arriving at the Golgi, proteins will face a much different environment than that of the ER (5) and will undergo different post translational modifications which have been extensively studied in the context of glycosylation (6). Glycoproteins will be extensively and distinctively modified in the Golgi, producing glycan microheterogeneity on individual glycoproteins. In immunoglobulin gamma (IgG) complexes, the heavy chain Fc regions are N-glycosylated and it has been shown that the microheterogeneity of these glycans can have an effect on protein structure (7-9) and function (10, 11). Differences in Fc chain glycan heterogeneity have been described as associated with aging, autoimmune disease, infectious disease, cancer and even pregnancy (12-16). The differences in environment and post-translational modification between the ER and the Golgi could potentially cause proteins that have been deemed properly folded in the ER, to be recognized as incorrectly folded or improperly modified in the Golgi. This possibility raises a requirement for a quality control mechanism in the Golgi which would be capable of distinguishing properly folded and modified proteins from misfolded and mis-modified ones, allowing the first to be secreted while targeting the latter for degradation. While Golgi stress has been postulated in the past (17), no direct evidence has been presented that correlates with Golgi associated quality control (GQC) or Golgi associated degradation (GAD). Ubiquitylation of Golgi proteins has been shown to occur and is attributed to regulation of Golgi membrane dynamics in the cell cycle (18), cis-Golgi integrity (19) and trafficking (20-22). Degradation of Golgi proteins by trafficking to the vacuole has recently been described in starvation conditions in yeast (23) but no evidence exists today of steady state proteasomal degradation in the Golgi.

### SUMMARY OF THE INVENTION

According to an aspect of some embodiments of the present invention there is provided a method of modulating protein exocytosis, the method comprising contacting a cell with an agent that modulates the ubiquitin pathway in the Golgi, thereby modulating protein secretion.

According to some embodiments of the invention, the protein exocytosis is selected from the group consisting of protein secretion, protein presentation on the plasma membrane, protein glycosylation.

According to an aspect of some embodiments of the present invention there is provided a method of inducing cell death, the method comprising contacting a cell having an aberrant Golgi quality control (GQC) machinery with an agent that modulates the ubiquitin pathway in the Golgi, thereby inducing the death of the cell.

According to some embodiments of the invention, the protein is a viral protein.

According to some embodiments of the invention, the cell death is mediated by inhibition of Golgi assembly.

According to some embodiments of the invention, the cell death is mediated by intracellular protein accumulation.

According to an aspect of some embodiments of the present invention there is provided a method of reconstituting normal GQC in a cell having aberrant GQC machinery, the method comprising contacting the cell with an agent that reconstitutes the GQC.

According to some embodiments of the invention, the aberrant GQC machinery is selected from the group consisting of aberrant Golgi ubiquitination machinery, aberrant secretion machinery, aberrant sorting machinery and aberrant glucosylation machinery.

According to some embodiments of the invention, the cell comprises an aberrant Golgi protein selected from the group of proteins listed in Table 1.

According to an aspect of some embodiments of the present invention there is provided a method of increasing protein degradation, the method comprising contacting a cell with an agent that inhibits transport of proteins through the Golgi, thereby increasing protein degradation.

According to some embodiments of the invention, the cell is a pathogenic cell.

According to some embodiments of the invention, the pathogenic cell is selected from the group consisting of a cancer cell, an immune cell and an infected cell.

According to some embodiments of the invention, the cell is a human cell.

According to an aspect of some embodiments of the present invention there is provided a method of treating a pathogenic condition associated with a secreted or membrane presented protein, the method comprising administering to a subject in need thereof an agent that modulates the GQC machinery, thereby treating the pathogenic condition associated with the aberrant protein exocytosis.

According to some embodiments of the invention, the agent modulates the ubiquitin pathway in the Golgi.

According to some embodiments of the invention, the agent that modulates the ubiquitin pathway in the Golgi upregulates activity of the ubiquitin pathway in the Golgi.

According to some embodiments of the invention, the agent that modulates the ubiquitin pathway in the Golgi downregulates activity of the ubiquitin pathway in the Golgi.

According to some embodiments of the invention, the agent modulates the activity or expression of a component of the ubiquitin pathway in the Golgi.

According to some embodiments of the invention, the component is selected from the group consisting of an E1 (Ubl), E2, E3, a proteasome subunit, a heat shock protein, a PHD containing protein, a deunbiquitinating enzyme and a regulator of any one of same.

According to some embodiments of the invention, the component is selected from the group of proteins listed in FIG. 1C.

According to some embodiments of the invention, the agent modulates protein secretion through the Golgi.

According to some embodiments of the invention, the agent that modulates protein secretion the Golgi is an inhibitor of protein secretion through the Golgi.

According to some embodiments of the invention, the agent that modulates protein secretion the Golgi is an inhibitor of protein secretion through the Golgi.

According to some embodiments of the invention, the agent inhibits COPII anterograde trafficking from endothelial reticulum (ER) to the Golgi.

According to some embodiments of the invention, the agent is H89.

According to some embodiments of the invention, the agent alters morphology of the Golgi.

According to some embodiments of the invention, the agent is megalomicin.

According to some embodiments of the invention, the agent inhibits glycosylation.

According to some embodiments of the invention, the agent inhibits sialyltransferase.

According to some embodiments of the invention, the agent is lythocholyglycine.

According to some embodiments of the invention, the condition is a pathogenic infection.

According to some embodiments of the invention, the condition is cancer.

According to some embodiments of the invention, the cancer is multiple myeloma (MM).

According to some embodiments of the invention, the agent is an inhibitor of protein secretion through the Golgi.

According to some embodiments of the invention, the agent is monensin.

According to some embodiments of the invention, the condition is an autoimmune disease.

According to some embodiments of the invention, the autoimmune disease is Systemic Lupus Erythematosus.

According to some embodiments of the invention, the condition is an amyloid disease.

According to some embodiments of the invention, the condition is an inflammatory disease.

According to some embodiments of the invention, the condition is a neurodegenerative disease.

According to some embodiments of the invention, the condition is associated with aging.

According to some embodiments of the invention, the condition is a congenital Golgi disease (CGD).

According to some embodiments of the invention, the condition is associated with cell senescence.

According to some embodiments of the invention, the contacting or administering comprises an effective amount for affecting the cell in a specific manner.

According to some embodiments of the invention, the subject is a human subject.

According to an aspect of some embodiments of the present invention there is provided a method of diagnosing a medical condition, the method comprising analyzing activity or expression of the GQC machinery in a subject in need thereof, wherein an aberrant activity or expression of the GQC in the subject is indicative of a medical condition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C depict systemic mapping of PTMs in Golgi localized proteins. (A) Graphical representations of PTM diversification in the Golgi apparatus and ER, highlighting the percentage of ubiquitylated proteins (red) in each organelle. Localization data was obtained from the human protein atlas and PTM data from PTMcode 2. (B) Functional classification of Golgi localized, ubiquitylated proteins. GO-terms were assigned by PANTHER classification system. (C) List of proteins, localized to the Golgi, that contain ubiquitin associated domains.

FIG. 2 shows that ubiquitylated proteins in the Golgi have roles in many diverse cellular pathways. Interaction map of Golgi localized, ubiquitylated, proteins and pathways to which they are associated. Size of circles is indicative of the P-value of pathways in relation to their general abundance in the cell.

FIGS. 3A-G show that proteotoxic stress induces significant changes in the polyubiquitylation of proteins in the Golgi. (A) Immunofluorescence images of HeLa cells either untreated or treated with the proteasomal inhibitor MG-132 (40 μM for 2 hrs) and stained for Golgi (giantin, green), polyubiquitin (red) and Nuclei (Hoechst, blue). (B) Quantification of immunofluorescent images showing the fold increase in intensity of polyubiquitin in the Golgi. Quantification of immunofluorescent images showing the mean intensity of polyubiquitin in the Golgi, in arbitrary units×10⁵ of untreated cells and cells treated with the proteasomal inhibitor MG-132. N=653, Pvalue=2.47×10⁻⁴⁶ (C) Immunofluorescence as in A for cells treated with tunicamycin (X μM for 2 hrs), Monensin (X μM for 2 hrs), Swainsonine (X μM for 2 hrs). (D) Quantification of images, showing the mean intensity of polyubiquitin in the Golgi of treated cells, normalized to untreated cells (fc=fold change). Average n=1,470, Pvalue(tunicamycin)=1.75×10⁻⁵⁸, Pvalue(monensin)=2.6×10⁻⁴⁰, Pvalue(swainsonine)=5.2×10⁻²⁷. (E) Western blots of fractions obtained from sucrose cushion centrifugation showing the separation achieved between Golgi (β-COP, TGN46), ER (calnexin), and Nuclei (Lamin A+C). (F) Ubiquitylation activity assay carried out in Golgi purified fractions over 0, 30 and 60 minutes, quantification normalized to 0 min at bottom. (G) Western blot against K-48 linked polyubiquitin chains in Golgi fractions from untreated cells and cells treated with proteotoxic stressors, quantification normalized to untreated cells at bottom.

FIG. 4 is a schematic representation of Golgi fraction purification. Schematic representation of the process of Golgi purification used for biochemical assays. Cells are grown in 4 15 cm plates, scraped and homogenized by dounce homogenizer with 0.5M sucrose in 100 mM HEPES pH 6.4. Homogenate is centrifuged for 10 min at 1,000 G and supernatant is loaded onto 0.86M sucrose in 100 mM HEPES pH 6.4. Sucrose cushion is centrifuged for 1 hr at 28,000 RPM in SW.41 rotor. Resulting gradient is fractionated and run on SDS-PAGE for analysis.

FIGS. 5A-C show activity assays for ER/Golgi fractions with various proteotoxic stresses. (A) Western blot of ER and Golgi fractions incubated with different components required for the ubiquitylation activity assay using HA-tagged ubiquitin and anti-HA antibody. Quantifications of polyubiquitin signal are shown as fold increase from the activity in the ER. (B) Western blots of Golgi fractions from untreated cells and cells treated with various proteotoxic stressors incubated over time (indicated) with HA-tagged ubiquitin and blotted with anti-HA antibody. Individual quantifications are shown as fold increase from 0 minutes of incubation. (C) Quantification of the increase in polyubiquitylation over time in western blots from B.

FIGS. 6A-G show that the Golgi contains a specific proteasomal subunit. (A) Immunofluorescence images of HeLa cells stained against Golgi (β-COP, green), the proteasomal subunit PSMD6 (red) and nuclei (Hoechst, blue). (B) Scanning electron microscopy images of purified Golgi fractions stained with immune-gold against PSMD6 and primary antibody control. (C) Western blot showing localization of PSMD6 and α6 proteasomal subunits across Golgi-ER fractionated cells. (D) Fluorescence images of HeLa cells expressing ts045 VSVG-GFP, grown at 40° C. and incubated at the permissive temperature of 32° C. for the stated times, transfected with either control siRNA or siRNA targeting PSMD6 (Golgi's are outlined in white). (E) Immunofluorescence images of HeLa cells stained for polyubiquitin, under the same conditions as in A. (F) Graph quantification of the increase in VSVG-GFP intensity in the Golgi (Grey bars) and increase in polyubiquitin intensity in the Golgi (black line) in HeLa cells transfected with control siRNA and grown at 40° C. and incubated at 32° C. for differential times. (G) Graph quantification as in F, of HeLa cells transfected with siRNA targeting PSMD6.

FIGS. 7A-D show that PSMD6 levels do not change in response to proteotoxic stress. (A) Immunofluorescence images of HeLa cells stained against a Golgi marker (β-COP, green) and PSMD6 (red), treated with proteotoxic stressors. (B) Quantification of mean PSMD6 intensities in Golgis of cells treated with Tunicamycin (n=7,397 Pvalue=5.02×10⁻⁴⁹), Monensin (n=5,778 Pvalue=0), Swainsonine (n=7,642 Pvalue=5.88×10⁻⁵¹) and MG-132 (n=6,974 Pvalue=8.78×10⁻⁶¹), normalized to untreated cells (n=9,681) (fc=fold change). (C) Quantification of mean PSMD6 intensities in whole cells of cells treated with Tunicamycin (n=7,397 Pvalue=1.75×10⁻⁰⁶), Monensin (n=5,778 Pvalue=1.4×10⁻²⁹⁹), Swainsonine (n=7,642 Pvalue=6.3×10⁻¹⁹⁹) and MG-132 (n=6,974 Pvalue=4.13×10⁻²³³), normalized to untreated cells (n=9,681) (fc=fold change). (D) Western blot showing PSMD6 levels in Golgi fractions and whole cell homogenates of cells either untreated or treated with proteotoxic stressors. Quantifications normalized to untreated cells at bottom.

FIGS. 8A-E show that the Golgi apparatus is capable of protein degradation and response to proteotoxic stress. (A) Schematic representation of the Suc-LLVY-AMC proteasomal degradation assay. (B) (C) Quantification of the fold increase in AMC fluorescence at the final measurement (t=150) from the initial time-point (t=0). Pvalue(untreated)=0.002, Pvalue(tunicamycin)=0.008, Pvalue(monensin)=0.01, Pvalue(swainsonine)=0.02, Pvalue(MG-132)=0.002. (D) Quantification of Proteasomal degradation in Golgi fractions. (E) Western blot against PSMD6 in cells transfected with control siRNA and siRNA against PSMD6.

Immunofluorescence images of HeLa cells treated with tunicamycin and stained against Golgi (β-COP, green), the proteasomal subunit PSMD6 (red) and nuclei (Hoechst, blue).

FIG. 9 shows that monensin treatment causes cell death. Various cell lines treated with 2 μM of monensin over 2 days show increased cell death. Cells were counted using countess 2 automated cell counter to determine live/dead ratios.

FIGS. 10A-F show apoptosis measurement by FACS analyses of murine MM 5TGM1 cells treated with monensin. The results are comparable to the treatment with bortezomib. Bortezomib is a golden standard drug for MM.

FIGS. 11A-D are graphs depicting that Monensin and bortezomib show comparable effects in killing MM cells.

FIGS. 12A-C show siRNA-mediated downregulation of PSMD6 and HACE1 synergistically sensitizes HeLa cells to both monensin and bortezomib.

FIGS. 13A-J show that Golgi stress provides a therapeutic opportunity in multiple myeloma. (A) Quantification of live/dead cells following 48 hours of monensin treatment (2 μM). (B) Quantification of cell death of RPMI-8226 cells over 3 days of treatment with monensin (2 μM). (C) Western blot against K48-linked polyubiquitin chains of Golgi fractions collected from RPMI 8226 cells either untreated or treated with monensin for 2 hrs. (D) XTT assay of untreated HeLa cells following siRNA mediated knockdowns as indicated Pvalue(siPSMD6+siHACE1)=0.002. (E) As in D, treated for 48 hours with monensin (200 nM) Pvalue(siPSMD6+siHACE1)=0.001. (F) Schematic outline of in-vivo experiment. (G) ELISA results of IgG2β levels in blood of mice injected with MM 5TGM1 cells over a period of 32 days. (H) FACS analysis and quantification of multiple myeloma cells (MM) and normal B cells (BC) in spleens of control vs. monensin-treated mice Pvalue(MM monensin)=0.008. (I) FACS analysis and quantification of multiple myeloma cells (MM) and normal B cells (BC) in bone marrow of control vs. monensin-treated mice. Pvalue(MM monensin)=0.001. (J) Spleen sizes of untreated, injected control vs. injected monensin-treated mice and uninjected, untreated mice.

FIGS. 14A-B show the effect of monensin in treatment of systemic lupus erythematosus. (A) MRL/LPR mice treated from 14 weeks old with 80 μM of monensin in drinking water, do not exhibit skin lesions that are characteristic of lupus. Furthermore, monensin treated mice were more relaxed when compared to mock (0.35% ethanol) treated mice. (B) Quantification of spleen weights of mock treated vs. monensin treated MRL/LPR mice as in A shows monensin treated spleens do not show an aberrant increase in weight characteristic of lupus. pValue<0.01.

### DESCRIPTION OF SPECIFIC EMBODIMENTS OF THE INVENTION

The present invention, in some embodiments thereof, relates to methods of modulating protein exocytosis and uses of same in therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Proteotoxicity is cell toxicity caused by proteins, usually of misfolded proteins but not exclusively. Any stress that perturbs homeostasis of proteins in the cell may be considered as proteotoxic (e.g. translation inhibitors, inhibitor of glycosylation enzymes, inhibitors of the proteasomes).

Proteotoxic stress leads to aberrant cellular processes and ultimately, apoptotic cell death. Quality control (QC) checkpoints assure the retention of misfolded proteins in subcellular organelles where these proteins can be probed and if need be, degraded by the ubiquitin proteasome system (UPS). The major quality control checkpoint in the cell has long been considered to take place in the endoplasmic reticulum (ER).

The present inventors have now uncovered a novel quality control checkpoint in the Golgi apparatus. Under proteotoxic stress, proteins are retained at the Golgi by a process of Golgi Quality Control (GQC). These proteins are then polyubiquitylated by Golgi-resident E3 ligases and degraded by a novel process termed Golgi-Associated Degradation (GAD). The ER QC checkpoint assures that proteins leaving the ER are properly folded, otherwise they will be retained for further folding attempts or degradation. The existence of GQC and GAD shows that a similar checkpoint exists in the Golgi and constitutes the final checkpoint for secretory proteins, preventing the secretion of aberrant proteins, which could lead to a potentially pathological state. Hence, harnessing the GQC and GAD mechanisms can be used for the development of novel therapeutic modalities.

Thus, every protein in the secretory pathway potentially undergoes GQC and the present inventors postulate that many pathological conditions can occur following perturbation of this pathway. By identifying and characterizing these perturbations, it is possible to target specific pathways either for remedy or for the elimination of cells with perturbed GQC capabilities. Examples for these uses are various.

Thus, according to an aspect of the invention there is provided a method of modulating protein exocytosis, the method comprising contacting a cell with an agent that modulates the ubiquitin pathway in the Golgi, thereby modulating protein secretion.

As used herein the phrase “protein exocytosis” refers to the exocytosis of soluble and non-soluble proteins. In other words the group of proteins relates to cell secreted proteins and to membrane anchored proteins. Generally the term relates to ATP-independent protein secretion. According to a specific embodiment, the protein is a secreted protein.

The term protein exocytosis is manifested by each or all (dependent on the nature of the protein) of protein secretion or protein presentation on the plasma membrane; and protein glycosylation.

As used herein “protein glycosylation” refers to glycosylation that occurs in the Golgi. The type of glycosylation typically depends on the type of cells used. For instance, plant cells have distinct glycosylation patterns when compared to mammalian cells.

Thus, the term typically refers to either O-linked glycosylation or to modification of N-linked glycans that occur exclusively in the Golgi.

As used herein “ubiquitin pathway in the Golgi” refers to the overall ubiquitin pathway in the Golgi, which may result in protein degradation. According to a specific embodiment, the pathway is composed of Golgi specific components which are not present in the ER or cytosol. Alternatively, the component of the Golgi ubiquitin pathway is not specific to the Golgi. In such a case, modulating this component is typically effected using a targeting moiety such as a moiety that binds a Golgi specific protein, not necessarily of the ubiquitin pathway. Examples of such Golgi specific targets include, but are not limited to, E3 ubiquitin protein ligases e.g., TRIM69, HECW2, CBX4, WWPI; Ubiquitin carboxyl terminal hydrolases e.g., USP7, USP8, USP32, UBAC1; PHD containing proteins e.g., TIF1A, VPS41, KAT6A, ING5, RNF214, ING2, ASH1L; UBLs e.g., CUL5, MED8, ZYG11B, WTC1, KLHL20, FBXW4; proteasomal subunits e.g., PSMD6, PSMB6.

According to a specific embodiment, the Golgi specific target is PSMD6 which is found in the Golgi membrane and hence serves as a good target for delivery.

According to a specific embodiment, the targeting moiety comprises an antibody (or any other affinity binding agent), as further described herein below.

An agent that modulates the ubiquitin pathway in the Golgi may be an agent, which downregulates or inhibits a component in the ubiquitin pathway in the Golgi, resulting in overall inhibition of protein degradation.

According to an alternative embodiment, the agent may upregulate, activate or increase the activity of a component in the ubiquitin pathway in the Golgi, resulting in overall increase in protein degradation.

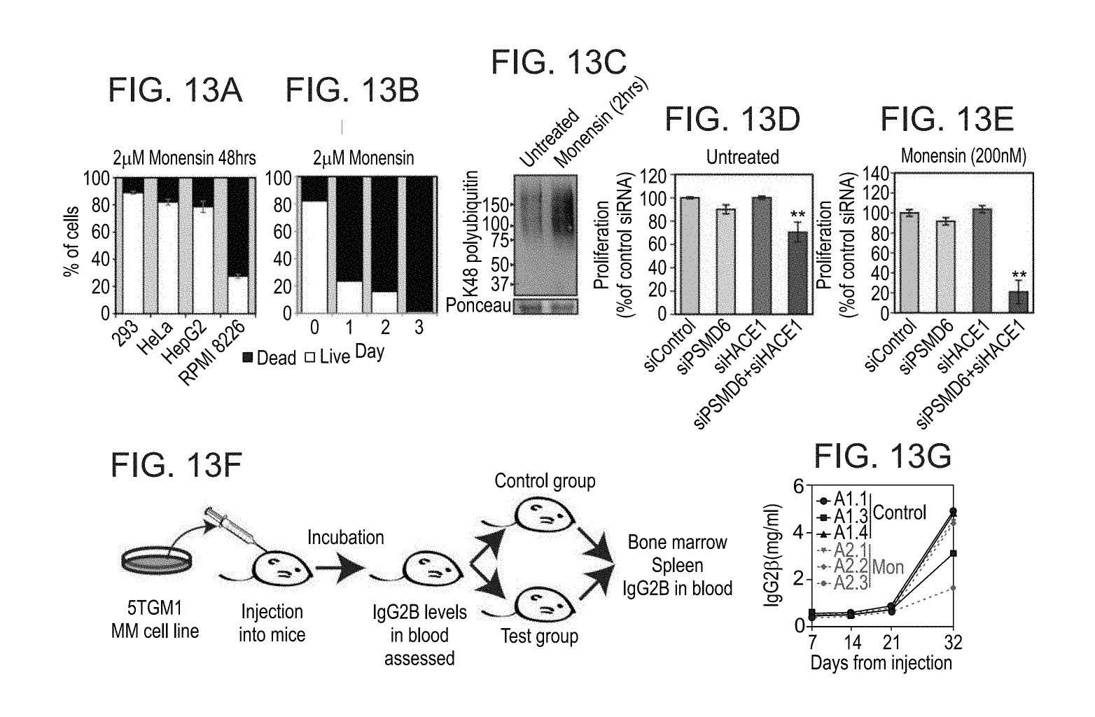

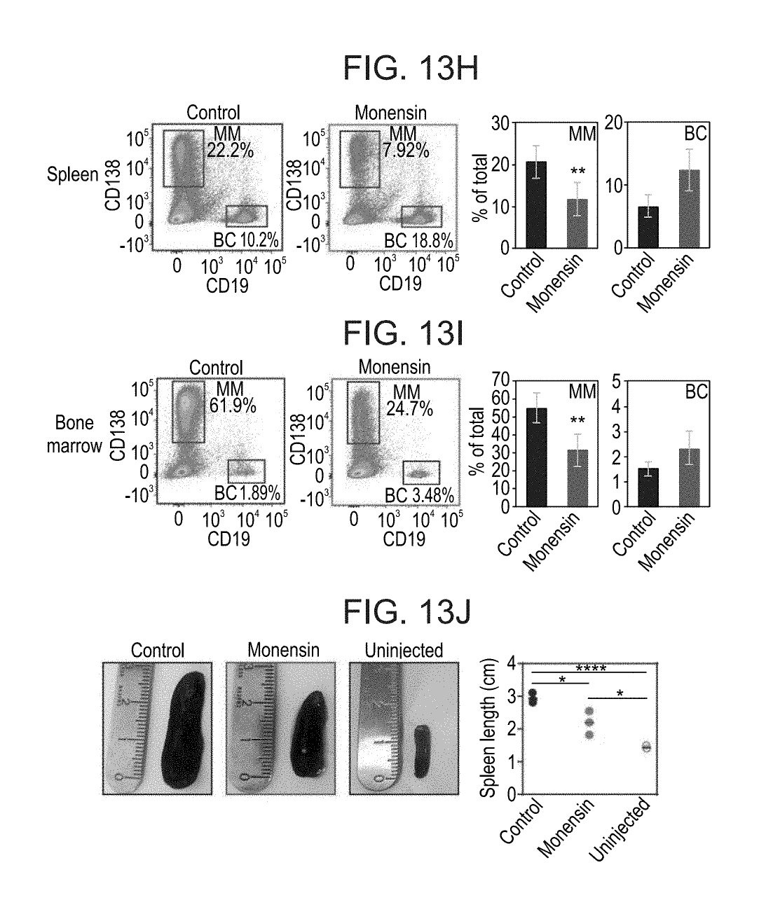

Without being bound by theory, it is suggested that perturbation of GQC e.g., by modulating GAD will result in cell death. It is further suggested that cells which are characterized by imbalanced protein secretion are specifically susceptible to this type of treatment, such as plasma cells (see FIGS. 13A-D).

Thus, it will be appreciated that any deviation of the GAD machinery will affect protein secretion (exocytosis) and cell viability, i.e., causing cell death.

Throughout the specification, downregulation, inhibition or decrease; upregulation, activation or increase, collectively termed “modulation”, refers to the statistically significant effect as compared to that in the absence of the agent under the same assay conditions.

In either case, the agent will affect the exocytosis and degradation of the protein in the cells either directly by influencing secretion machinery or indirectly by causing upregulation of transcription, translation or activation (e.g. via protein modification) of said machinery or other components of GQC such as glycosylation enzymes or chaperones.

According to a specific embodiment, the component is selected from the group consisting of an E1 (Ubl), E2, E3, a proteasome subunit, a heat shock protein, a PHD containing protein, a deubiquitinating enzyme and a regulator of any one of same.

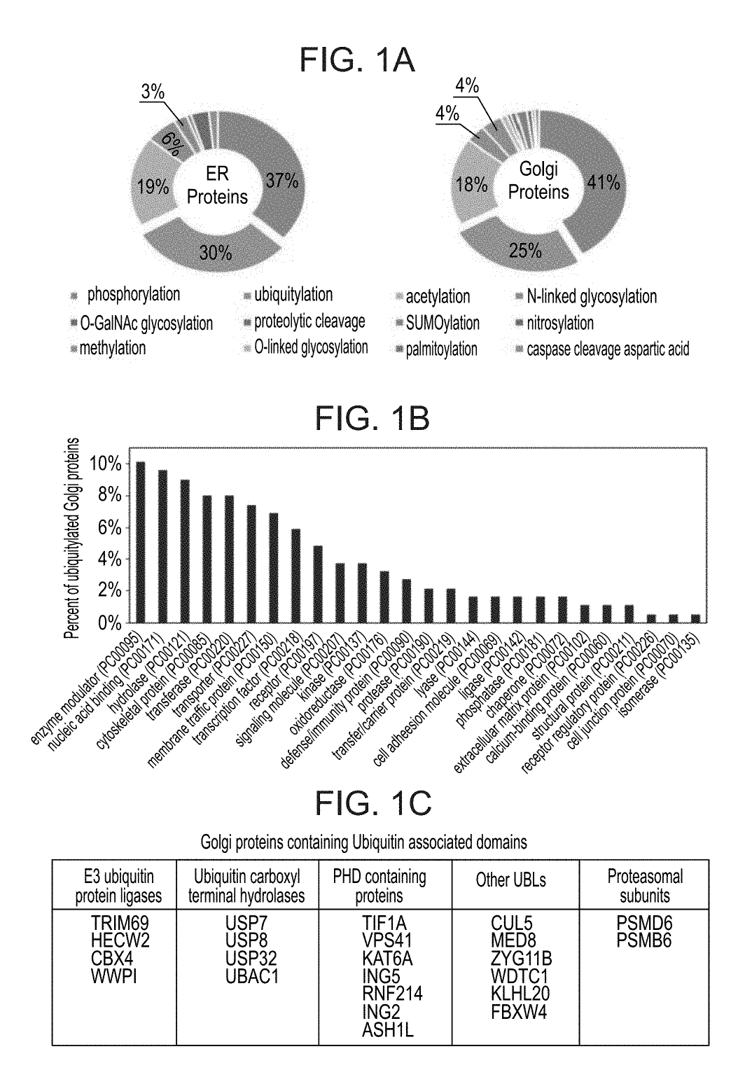

Specific components of the ubiquitin pathway in the Golgi include but are not limited to those listed in FIG. 1C and hereinabove.

Such components are present in the Golgi and not in another cellular localization (as determined at the protein level e.g., by quantitative immunofluorescence assays and Western blot analysis of subcellular fractions).

As mentioned, protein accumulation in the cells causes cytotoxicity, thus some of the above embodiments will eventually result in the induction of cell death. Alternatively, imbalanced increase in GAD causes cytotoxicity as well.

Thus, according to an aspect of the invention there is provided a method of inducing cell death, the method comprising contacting a cell having an aberrant Golgi quality control (GQC) machinery with an agent that modulates the ubiquitin pathway in the Golgi, thereby inducing the death of the cell.

Thus, according to some embodiments of the invention the agent may induce cell death by inhibition of Golgi assembly.

Both abnormal assembly and protein accumulation can be assessed by immunofluorescence of Golgi markers [(e.g. bCOP and labeled lectins or WGA), which show Golgi fragmentation and glycoprotein accumulation, respectively].

According to an alternative or an additional embodiment, the agent induces cell death by intracellular protein accumulation. In this case, it is evident that inhibition of protein degradation will result in accumulation and hence cell death.

Agents which upregulate or downregulate activity or expression of proteins are known in the art and are listed hereinbelow.

As the present invention is based on the new finding of GQC it is evident that where aberrant GQC is present, the cell is more susceptible to damage of the secretory pathway.

Accordingly, there is provided a method of inducing cell death, the method comprising contacting a cell having an aberrant Golgi quality control (GQC) machinery with an agent that modulates the ubiquitin pathway in the Golgi, thereby inducing the death of the cell.

As used herein “cell” refers to a eukaryotic cell which comprises the Golgi system. Examples include mammalian cells (e.g., human or non-human cells), plant cells, yeast cells, fungal cells, algal cells, insect cells. The cell can be a differentiated cell, a stem cell (e.g., embryonic stem cell, induced pluripotent stem cell, mesenchymal stem cell, hematopoietic stem cell, neural stem cell) or a progenitor cell.

According to a specific embodiment, the cell is a cell line.

According to a specific embodiment, the cell is a primary cell.

According to a specific embodiment, the cell is in a cell culture.

According to a specific embodiment, the cell forms a part of a tissue.

According to a specific embodiment, the cell forms a part of an organism.

According to a specific embodiment, the cell is a healthy cell (i.e., taken from an organism not affected with a disease e.g., the diseases listed below).

According to a specific embodiment, the cell is a pathogenic cell (affected with a disease).

According to a specific embodiment, the pathogenic cell is selected from the group consisting of a cancer cell, an immune cell and an infected cell, e.g., a viral or bacterial infected cell.

According to a specific embodiment, the cell is a secretory cell. According to a specific embodiment a secretory cell stems a secretory tissue such as liver, pancreas, bone marrow, CNS, blood and colon.

According to a specific embodiment, the cell is a pathogenic secretory cell, meaning that onset or progression of disease is associated with imbalanced protein (e.g., immunoglobulin) secretion such as in myeloma. Other examples are provided infra.

Examples of such cells include, but are not limited to, secretory cells such as viral infected cells, plasma cells, hepatocytes, cells of the digestive tract, hormone secreting cells, immune cells, adrenal gland cells and neurons.

Examples of secretory cells are provided in Table A below (secreted proteins are in parenthesis).

TABLE A

| | | |
|---|---|---|
| NCI-H295 Adrenal gland | HCC1569 Breast, primary | BDCM Leukemia, acute |
| adrenocortical carcinoma. | metaplastic carcinoma. (HER2, | myelogenous, B lymphoblast. |
| (Hormones, such as | VEGF) | (IL-6) |
| aldosterone; cortisol; C19 | | |
| steroids) | | |
| SW-13 Adrenal gland primary | Caco-2 Colon, adenocarcinoma | |
| small cell carcinoma. | (IL-6) | |
| (Hormones such as Endothelin | | |
| 1, adrenomedullin). | | |
| Hs 683 Brain glioma. (TGF-β) | SNU-C1 Colon, | |
| | adenocarcinoma | |
| | (carcinoembryonic | |
| | antigen) | |
| | C2BBe1l Colon, colorectal | Molt-4 Leukemia, T |
| | adenocarcinoma (IL-18, IL-8) | lymphoblast. (Prostaglandin E, |
| | | VEGF) |
| IMR-32 Brain, neuroblastoma | HCT-15 Colon, colorectal | HuH28 Liver, bile duct |
| (APP) | carcinoma (CD63, CD9, CD81) | carcinoma (ALP, GGT, BMG, |
| | | ferritin, elastase-1, TPA) |
| | | PLC/PRF/5 Liver, hepatoma |
| | | (Hepatitis B surface antigen) |
| MCF-7 Breast adenocarcinoma | Kasumi-3 Leukemia, actue | |
| (TGF-β2) | myeloblastic leukemia | |
| | (galectin 9) | |
| BT-474 Breast, ductal | SUP-B15 Leukemia, acute | QGP-1 Pancreas, pancreatic |
| carcinoma (HER2 and others) | lymphoblastic, B lymphoblast | islet cell carcinoma |
| | (IL-6, IL-10, antibodies) | (Somatostatin) |
| MDA-MB-157 Breast | | |
| medulallary carcinoma. | | |
| (Robo1) | | |
| HCC1806 Breast, primary | | |
| acantholytic squamous cell | | |
| carcinoma. (SFRP1) | | |

As used herein “cell death” refers to apoptosis dependent cell death.

Also provided is a method of reconstituting normal GQC in a cell having aberrant GQC machinery, the method comprising contacting the cell with an agent that reconstitutes said GQC.

As used herein “GQC” or “GQC machinery” refers to Golgi resident or non-Golgi resident proteins which are associated with GAD, glycosylation, secretion of proteins and/or the sensing of aberrant protein folding and/or glycosylation in the Golgi.

As used herein “normal GQC” the activity of the above GQC machinery in a normal cell (not affected with a disease).

As used herein “aberrant GQC” any imbalance in the activity (dysfunction) of the GQC as compared to that in a normal cell.

According to a specific embodiment, the cell having the aberrant GQC machinery comprises an aberrant Golgi protein such as listed in Table 1 below.

As explained above, GAD is directly linked to protein exocytosis, as such also provided is a method of increasing protein degradation. The method comprising contacting a cell (e.g., as described above) with an agent that inhibits transport of proteins through the Golgi, thereby increasing protein degradation.

Upregulation of a protein (e.g., a GQC component) can be effected at the genomic level (i.e., activation of transcription via promoters, enhancers, regulatory elements e.g., by genome editing), at the transcript level (i.e., correct splicing, polyadenylation, activation of translation) or at the protein level (i.e., post-translational modifications, interaction with substrates and the like).

Following is a list of agents capable of upregulating the expression level and/or activity of a protein.

An agent capable of upregulating expression of a protein may be an exogenous polynucleotide sequence designed and constructed to express at least a functional portion of the protein. Accordingly, the exogenous polynucleotide sequence may be a DNA or RNA sequence encoding a GQC protein, capable of modulating the GQC machinery.

The phrase “functional portion” as used herein refers to part of the GQC protein (i.e., a polypeptide) which exhibits functional properties of the enzyme (e.g., E3 ligase) such as binding to a substrate.

To express exogenous proteins in mammalian cells, a polynucleotide sequence encoding the protein is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of some embodiments of the invention can also utilize homologues which exhibit the desired activity (i.e., GQC). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to a protein of interest, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments of the invention are promoter sequences which are active under most environmental conditions and most types of cells such as the cytomegalovirus (CMV) and Rous sarcoma virus (RSV). Inducible promoters suitable for use with some embodiments of the invention include for example the inducible promoter of the tetracycline-inducible promoter (Zabala M, et al., Cancer Res. 2004, 64(8): 2799-804).

To express exogenous proteins in eukaryotic cells, a polynucleotide sequence encoding a protein of interest may be ligated into a nucleic acid construct suitable for eukaryotic cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Retention mechanisms employed by the glycosyltransferases/glycosidases and by the SNAREs, have been best characterized and the skilled artisan would know whether to use the proteins endogenous (native sequence) or to modify the coding sequence to include heterologous sequence for Golgi retention.

A number of mechanisms for Golgi retention are known in the art. These include, but are not limited to, oligomerization, TMD-based partitioning, COPI-mediated retrieval, lipid composition based partitioning and Vsp74p/GOLPH3-mediated retention. Examples of retention signals that can be employed according to the present teachings, are described in Banfield Cold Spring Harb Perspect Biol. 2011 August; 3(8): a005264, which is hereby incorporated by reference in its entirety.

Thus, the agent can be translationally fused to a Golgi retention signal so as to confer specificity to a Golgi non-specific agent. For example, the agent can be chemically/translationally fused to an affinity moiety or to a Golgi localization signal. The affinity moiety can be, for example, a transmembrane peptide, part of the galactosyltransferase enzyme (e.g., beta 1, 4, galactosyltranferase-1), which is commonly used to convey Golgi localization to chimeric proteins. The agent can be monensin, imparting a more specific Golgi-localized targeting of this drug.

It will be appreciated that the individual elements comprised in the expression vector can be arranged in a variety of configurations. For example, enhancer elements, promoters and the like, and even the polynucleotide sequence(s) encoding a protein or interest can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of a protein of interest since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses.

A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct.

Importantly, the coding sequence for expression of the protein of interest should include a Golgi retention signal to the extent that such a protein of interest does not already include an endogenous (naturally occurring) Golgi retention signal.

It will be appreciated that upregulation of a protein of interest can be also effected by administration of cells that express the protein of interest (e.g., a GQC component) into the individual.

An agent capable of upregulating GQC machinery can also be a small molecule e.g., which activates secretion.

Upregulation of a protein of interest can also be effected at the genomic level using genome editing techniques (described in length hereinbelow), designed to increase the activity of a promoter element (or other regulatory sequence which affects transcription for instance) or at the coding sequence level (increasing catalytic activity or protein binding activities).

Conversely, downregulation of a protein of interest (e.g., of a GQC machinery) may be effected at the protein level (down-regulating activity or affecting post-translational modifications), at the transcript level or at the genome level.

As used herein the phrase “dowregulates expression or activity” refers to dowregulating the expression of a protein at the genomic (e.g. genome editing) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating expression are described in details hereinbelow.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of expression can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., of a GQC machinery) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server. Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, suitable siRNAs directed against the target (e.g., GQC machinery component) can be the ones listed in the Examples section which follows.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA Mimics

According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense

Antisense is a single stranded RNA designed to prevent or inhibit expression of a gene by specifically hybridizing to its mRNA. Downregulation of expression of a protein of interest can be effected using an antisense polynucleotide capable of specifically hybridizing with an mRNA transcript encoding the protein of interest.

The prior art teaches of a number of delivery strategies which can be used to efficiently deliver oligonucleotides into a wide variety of cell types [see, for example, Jääskeläinen et al. (2002) 7(2):236-7; Gait, Cell Mol Life Sci. (2003) 60(5):844-53; et al. J Biomed Biotechnol. (2009) 2009:410260; et al. (2014) 24(7):801-19; Falzarano et al, Nucleic Acid Ther. (2014) 24(1):87-100; Shilakari et al. (2014) 2014: 526391; Prakash et al. Nucleic Acids Res. (2014) 42(13):8796-807 and Asseline et al. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Downregulation of expression of a protein of interest can also be achieved by inactivating the gene (e.g., of a GQC machinery) via introducing targeted mutations involving loss-of function alterations (e.g. point mutations, deletions and insertions) in the gene structure.

As used herein, the phrase “loss-of-function alterations” refers to any mutation in the DNA sequence of a gene, which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. gene encoding the GQC protein of interest may be in a homozygous form or in a heterozygous form. According to this embodiment, homozygosity is a condition where both alleles at the e.g. GQC machinery component locus are characterized by the same nucleotide sequence. Heterozygosity refers to different conditions of the gene at the e.g. GQC gene locus.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases

Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs

Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through http://www(dot)talendesign(dot)org). TALEN can also be designed and obtained commercially from e.g., Sangamo Biosciences™ (Richmond, Calif.).

CRISPR-Cas System

Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvik and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. Dec. 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Down-Regulation at the Polypeptide Level

According to specific embodiments the agent capable of downregulating a GQC protein is an antibody or antibody fragment capable of specifically binding the GQC protein. Preferably, the antibody specifically binds at least one epitope of a GQC protein in a specific manner. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complernentarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallumn et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule;

(iii) disulfide-stabilized Fv (“dsFv”), a genetically engineered antibody including the variable region of the light chain and the variable region of the heavy chain, linked by a genetically engineered disulfide bond;

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As the GQC machinery is intracellular, the antibody or antibody fragment capable of specifically binding the GQC component may be an intracellular antibody.

Alternatively or additionally a cell penetrating peptide (CPP) or formulations used for introducing the antibody (or polypeptides) or any other cellular agent as described herein to the cell may be used.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Another agent which can be used along with some embodiments of the invention to downregulate a component of the GQC machinery is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Alternatively or additionally, small molecule or peptides can be used which interfere with protein function (e.g., catalytic or interaction).

Small molecules for modulating secretion through the Golgi thus affecting GAD (and vise a versa) are well known in the art.

Following is a non-limiting list.
Inhibitors of protein secretion/transport through the Golgi.
Tunicamycin—Inhibits addition of UDP-GlcNAc to dolichol biphosphate, effectively inhibiting N-glycosylation in the ER.
Castanospermine—Inhibits the ER α-glucosidases, disrupting the QC of glycoproteins.
DNJ—Inhibits both the ER α-glucosidases and the Golgi α1,2 mannosidases.
Swainsonine—Inhibits Golgi mannosidase 2.
Indol—Inhibits the Golgi fucosyltransferase.
Brefeldin A—inhibitor of COPI retrograde trafficking from Golgi to ER.
H89—Inhibitor of COPII anterograde trafficking from ER to Golgi.
Monensin—Inhibitor of intra-Golgi trafficking from medial to trans.
Megalomicin—Alters Golgi morphology, inhibits intra-Golgi trafficking.

According to a specific embodiment, the agent is selected from the group consisting of monensin, megalomicin, LCG and H89.

According to a specific embodiment, the agent is selected from the group consisting of megalomicin, LCG and H89.

According to a specific embodiment, the agent is monensin.

Agents that can be used in accordance with the present teachings can be qualified using a cell based assay, such as a cell viability assay. For example, the XTT cell viability assay is based on cellular metabolism. This assay utilizes NADH, produced in mitochondria of live cells only, to reduce XTT molecules to form a colored compound which can then be gauged by plate reader. Output of this assay, when paired with a standard curve, is number of viable cells per well of 96-well plate. Different cell types are plated and treated in triplicates before being exposed to XTT, parallel to standard curve plating of known amounts of untreated cells in triplicates per cell type.

The present teachings can be harnessed towards clinical applications (therapy and diagnostics).

Therapy

Thus, according to an aspect of the invention there is provided a method of treating a pathogenic condition associated with a secreted or membrane presented protein, the method comprising administering to a subject in need thereof an agent that modulates the GQC machinery, thereby treating the pathogenic condition associated with the aberrant protein exocytosis.

As used herein “an agent that modulates the GQC machinery” refers to an agent that either restores the presentation (secretion) of the protein, downregulates secretion of the protein, restores the cells ability to identify and effectively degrade the protein or that kills the cell expressing same (such agents are described hereinabove, e.g., small molecules such as monensin, H89, Megalomicin and lithocholyglycine).

Following is a list of diseases which are associated with a secreted or membrane presented protein

TABLE 1

| Muscle Eye Brain Disease | Neuronal diseases, Muscle diseases, Eye | LARGE, |
|---|---|---|
| | diseases, Genetic diseases, Metabolic | POMGNT1, |
| | diseases, Rare diseases, Fetal diseases | POMT1, |
| | | POMT2, FKRP |
| Congenital Muscular | Neuronal diseases, Muscle diseases, | FKRP, LARGE, |
| Dystrophy with Intellectual | Metabolic diseases, Rare diseases | POMT1, |
| Disability | | POMT2 |
| Congenital Muscular | Neuronal diseases, Muscle diseases, | POMT1, |
| Dystrophy with Cerebellar | Metabolic diseases, Rare diseases | POMT2, |
| Involvement | | POMGNT1, |
| | | FKRP |
| Walker-Warburg Syndrome | Neuronal diseases, Muscle diseases, Eye | FKRP, POMT1, |
| | diseases, Genetic diseases, Metabolic | POMT2, |
| | diseases, Rare diseases, Fetal diseases | LARGE, |
| | | POMGNT1 |
| Muscular Dystrophy- | Neuronal diseases, Muscle diseases, Mental | FKRP, |
| Dystroglycanopathy | diseases, Genetic diseases | POMGNT1, |
| | | LARGE, |
| | | POMT2, |
| | | POMT1 |
| Exostoses, Multiple, Type 1 | Bone diseases, Genetic diseases, Metabolic | EXT1, EXT2 |
| | diseases, Rare diseases, Fetal diseases, | |
| | Cancer diseases | |
| Congenital Muscular | Neuronal diseases, Muscle diseases, | FKRP, POMT1 |
| Dystrophy Without Intellectual | Metabolic diseases, Rare diseases | |
| Disability | | |
| Brain Disease | Neuronal diseases | FKRP, LARGE, |
| | | POMGNT1, |
| | | POMT1 |
| Glycosylphosphatidylinositol | Respiratory diseases, Genetic diseases, Rare | PIGM, PIGV |
| Deficiency | diseases | |
| Congenital Disorder of | Neuronal diseases, Muscle diseases, Blood | COG7, COG1 |
| Glycosylation, Type Ii | diseases, Liver diseases, Genetic diseases, | |
| | Metabolic diseases, Rare diseases | |
| Congenital Disorder of | Neuronal diseases, Muscle diseases, Blood | COG8, COG6 |
| Glycosylation, Type Iih | diseases, Liver diseases, Genetic diseases, | |
| | Metabolic diseases, Rare diseases | |
| Hereditary Multiple Exostoses | Bone diseases, Genetic diseases, Metabolic | EXT2, EXT1 |
| | diseases, Rare diseases, Fetal diseases, | |
| | Cancer diseases | |
| Muscular Dystrophy | Neuronal diseases, Muscle diseases, | FKRP, POMT1, |
| | Cardiovascular diseases, Genetic diseases, | LARGE |
| | Metabolic diseases, Rare diseases | |
| Hereditary Multiple | Bone diseases, Genetic diseases, Rare | EXT2, EXT1 |
| Osteochondromas | diseases | |
| Peters Anomaly | Neuronal diseases, Eye diseases, | B3GALTL, |
| | Cardiovascular diseases, Genetic diseases, | EXT1 |
| | Metabolic diseases, Rare diseases, Fetal | |
| | diseases | |
| Dysplasia Epiphysealis | Bone diseases, Rare diseases, Fetal diseases | EXT1, EXT2 |
| Hemimelica | | |
| Muscular Dystrophy- | Neuronal diseases, Muscle diseases, Mental | FKRP |
| Dystroglycanopathy, Type B, 5 | diseases, Genetic diseases | |
| Muscular Dystrophy- | Neuronal diseases, Muscle diseases, Mental | LARGE |
| Dystroglycanopathy, Type B, 6 | diseases, Genetic diseases | |
| Muscular Dystrophy- | Neuronal diseases, Muscle diseases, Mental | POMT2 |
| Dystroglycanopathy, Type C, 2 | diseases, Genetic diseases | |
| Hyperphosphatasia with | Neuronal diseases, Mental diseases, Genetic | PIGV |
| Mental Retardation Syndrome | diseases | |
| 1 | | |
| Hypercoagulability Syndrome | Neuronal diseases, Blood diseases, Metabolic | PIGM |
| Due to | diseases, Rare diseases | |
| Glycosylphosphatidylinositol | | |
| Deficiency | | |
| Spondyloepiphyseal Dysplasia | Bone diseases, Genetic diseases, Rare | CHST3 |
| with Congenital Joint | diseases | |
| Dislocations | | |
| Muscular Dystrophy- | Neuronal diseases, Muscle diseases, Mental | FKRP |
| Dystroglycanopathy, Type a, 5 | diseases, Genetic diseases | |
| Schneckenbecken Dysplasia | Bone diseases, Genetic diseases, Metabolic | SLC35D1 |
| | diseases, Rare diseases, Fetal diseases | |
| Muscular Dystrophy- | Neuronal diseases, Muscle diseases, Mental | POMT2 |
| Dystroglycanopathy, Type a, 2 | diseases, Genetic diseases | |
| Congenital Disorder of | Neuronal diseases, Muscle diseases, Blood | COG6 |
| Glycosylation, Type Iil | diseases, Liver diseases, Genetic diseases, | |
| | Metabolic diseases, Rare diseases | |
| Autosomal Recessive Limb- | Neuronal diseases, Muscle diseases, Genetic | POMGNT1 |
| Girdle Muscular Dystrophy | diseases, Metabolic diseases, Rare diseases | |
| Type 2o | | |
| Tumoral Calcinosis, | Genetic diseases, Rare diseases, Cancer | GALNT3 |
| Hyperphosphatemic, Familial | diseases | |
| Wrinkly Skin Syndrome | Neuronal diseases, Eye diseases, Bone | ATP6V0A2 |
| | diseases, Skin diseases, Genetic diseases, | |
| | Metabolic diseases, Rare diseases, Fetal | |
| | diseases | |
| Dyserythropoietic Anemia, | Blood diseases, Genetic diseases, Metabolic | SEC23B |
| Congenital, Type Ii | diseases, Rare diseases | |
| Congenital Disorder of | Neuronal diseases, Muscle diseases, Blood | COG4 |
| Glycosylation, Type Iij | diseases, Liver diseases, Genetic diseases, | |
| | Metabolic diseases, Rare diseases | |
| Muscular Dystrophy- | Neuronal diseases, Muscle diseases, Mental | LARGE |
| Dystroglycanopathy, Type a, 6 | diseases, Genetic diseases | |
| Autosomal Recessive Limb- | Neuronal diseases, Muscle diseases, Genetic | POMT2 |
| Girdle Muscular Dystrophy | diseases, Metabolic diseases, Rare diseases | |
| Type 2n | | |
| Autosomal Recessive Limb- | Neuronal diseases, Muscle diseases, Genetic | FKRP |
| Girdle Muscular Dystrophy | diseases, Metabolic diseases, Rare diseases | |
| Type 2i | | |
| Ehlers-Danlos Syndrome, | Neuronal diseases, Muscle diseases, Bone | CHST14 |
| Musculocontractural Type 1 | diseases, Cardiovascular diseases, Skin | |
| | diseases, Nephrological diseases, Genetic | |
| | diseases, Metabolic diseases, Rare diseases, | |
| | Fetal diseases | |
| Muscular Dystrophy- | Neuronal diseases, Muscle diseases, Mental | POMGNT1 |
| Dystroglycanopathy, Type C, 3 | diseases, Genetic diseases | |
| Ehlers-Danlos Syndrome, | Bone diseases, Skin diseases, Genetic | B4GALT7 |
| Progeroid Type, 1 | diseases, Metabolic diseases, Rare diseases, | |
| | Fetal diseases | |
| Ehlers-Danlos Syndrome | Bone diseases, Skin diseases, Genetic | B4GALT7 |
| Progeroid Type | diseases, Metabolic diseases, Rare diseases, | |
| | Fetal diseases | |
| Muscular Dystrophy- | Neuronal diseases, Muscle diseases, Mental | POMGNT1 |
| Dystroglycanopathy, Type a, 3 | diseases, Genetic diseases | |
| Muscular Dystrophy- | Neuronal diseases, Muscle diseases, Mental | POMT1 |
| Dystroglycanopathy, Type a, 1 | diseases, Genetic diseases | |
| Congenital Disorder of | Neuronal diseases, Muscle diseases, Blood | MGAT2 |
| Glycosylation, Type Iia | diseases, Liver diseases, Genetic diseases, | |
| | Metabolic diseases, Rare diseases | |
| Hypohidrosis-Enamel | Neuronal diseases, Skin diseases, Oral | COG6 |
| Hypoplasia-Palmoplantar | diseases, Rare diseases | |
| Keratoderma-Intellectual | | |
| Disability Syndrome | | |
| Congenital Disorder of | Neuronal diseases, Muscle diseases, Blood | B4GALT1 |
| Glycosylation, Type Iid | diseases, Liver diseases, Genetic diseases, | |
| | Metabolic diseases, Rare diseases | |
| Reunion Island's Larsen | Bone diseases, Rare diseases, Fetal diseases | B4GALT7 |
| Syndrome | | |
| Congenital Disorder of | Neuronal diseases, Muscle diseases, Blood | SLC35A1 |
| Glycosylation, Type Iif | diseases, Liver diseases, Genetic diseases, | |
| | Metabolic diseases, Rare diseases | |
| Exostoses, Multiple, Type 2 | Bone diseases, Genetic diseases, Metabolic | EXT2 |
| | diseases, Rare diseases, Fetal diseases, | |
| | Cancer diseases | |
| Wieacker-Wolff Syndrome | Neuronal diseases, Muscle diseases, Genetic | POMT1 |
| | diseases, Rare diseases, Fetal diseases | |
| Craniolenticulosutural | Bone diseases, Genetic diseases, Rare | SEC23A |
| Dysplasia | diseases, Fetal diseases | |
| Congenital Disorder of | Neuronal diseases, Muscle diseases, Blood | COG1 |
| Glycosylation, Type Iig | diseases, Liver diseases, Genetic diseases, | |
| | Metabolic diseases, Rare diseases | |
| Autosomal Recessive Cutis | Neuronal diseases, Eye diseases, Bone | ATP6V0A2 |
| Laxa Type 2, Classic Type | diseases, Skin diseases, Metabolic diseases, | |
| | Rare diseases, Fetal diseases | |
| Shaheen Syndrome | Genetic diseases | COG6 |
| Muscular Dystrophy- | Neuronal diseases, Muscle diseases, Mental | POMT2 |
| Dystroglycanopathy, Type B, 2 | diseases, Genetic diseases | |
| Macular Corneal Dystrophy | Eye diseases, Genetic diseases, Rare diseases | CHST6 |
| Cutis Laxa, Autosomal | Neuronal diseases, Eye diseases, Bone | ATP6V0A2 |
| Recessive, Type Iia | diseases, Cardiovascular diseases, | |
| | Gastrointestinal diseases, Skin diseases, | |
| | Nephrological diseases, Genetic diseases, | |
| | Metabolic diseases, Rare diseases, Fetal | |
| | diseases | |
| Muscular Dystrophy- | Neuronal diseases, Muscle diseases, Mental | POMGNT1 |
| Dystroglycanopathy, Type B, 3 | diseases, Genetic diseases | |
| Autosomal Recessive Limb- | Neuronal diseases, Muscle diseases, Genetic | POMT1 |
| Girdle Muscular Dystrophy | diseases, Metabolic diseases, Rare diseases | |
| Type 2k | | |
| Muscular Dystrophy- | Neuronal diseases, Muscle diseases, Mental | POMT1 |
| Dystroglycanopathy, Type B, 1 | diseases, Genetic diseases | |
| Muscular Dystrophy- | Neuronal diseases, Muscle diseases, Mental | POMT1 |
| Dystroglycanopathy, Type C, 1 | diseases, Genetic diseases | |
| Congenital Disorder of | Neuronal diseases, Muscle diseases, Blood | COG5 |
| Glycosylation, Type Iii | diseases, Liver diseases, Genetic diseases, | |
| | Metabolic diseases, Rare diseases | |
| Potocki-Shaffer Syndrome | Genetic diseases, Rare diseases, Fetal | EXT2 |
| | diseases | |
| Trichorhinophalangeal | Neuronal diseases, Bone diseases, Skin | EXT1 |
| Syndrome, Type Ii | diseases, Genetic diseases, Rare diseases, | |
| | Fetal diseases | |
| Hyperphosphatasia-Intellectual | Neuronal diseases, Mental diseases, Bone | PIGV |
| Disability Syndrome | diseases, Metabolic diseases, Rare diseases, | |
| | Fetal diseases | |
| Ollier Disease | Bone diseases, Genetic diseases, Rare | EXT1, EXT2 |
| | diseases, Fetal diseases, Cancer diseases | |
| Osteochondroma | Bone diseases, Genetic diseases, Rare | EXT1, EXT2 |
| | diseases | |
| Exostosis | Bone diseases | EXT2, EXT1 |
| Familial Tumoral Calcinosis | Skin diseases, Endocrine diseases, Genetic | GALNT3 |
| | diseases, Metabolic diseases, Rare diseases, | |
| | Cancer diseases | |
| Hereditary Multiple | Bone diseases, Genetic diseases | EXT1 |
| Osteochondromatosis, Type I | | |
| Hyperphosphatasia with | Neuronal diseases, Mental diseases, Genetic | PIGV |
| Mental Retardation Syndrome | diseases | |
| Hereditary Multiple | Bone diseases, Genetic diseases | EXT2 |
| Osteochondromatosis, Type Ii | | |
| Pomt2-Related Muscle | Neuronal diseases, Genetic diseases | POMT2 |
| Diseases | | |
| Large-Related Muscle Diseases | Neuronal diseases, Genetic diseases | LARGE |
| Fkrp-Related Muscle Diseases | Neuronal diseases, Genetic diseases | FKRP |
| Pomgnt1-Related Muscle | Neuronal diseases, Genetic diseases | POMGNT1 |
| Diseases | | |
| Pomt1-Related Muscle | Neuronal diseases, Genetic diseases | POMT1 |
| Diseases | | |
| Atp6v0a2-Related Cutis Laxa | Neuronal diseases, Eye diseases, Bone | ATP6V0A2 |
| | diseases, Cardiovascular diseases, | |
| | Gastrointestinal diseases, Skin diseases, | |
| | Nephrological diseases, Genetic diseases, | |
| | Metabolic diseases, Rare diseases, Fetal | |
| | diseases | |
| Hyperphosphatemic Familial | Genetic diseases, Rare diseases, Cancer | GALNT3 |
| Tumoral Calcinosis, Galnt3- | diseases | |
| Related | | |
| Larsen Syndrome, Autosomal | Bone diseases, Genetic diseases, Rare | CHST3 |
| Recessive | diseases, Fetal diseases | |
| Chondrosarcoma | Bone diseases, Genetic diseases, Rare | EXT1 |
| | diseases, Cancer diseases | |
| Muscular Dystrophy- | Neuronal diseases, Muscle diseases, Mental | FKRP, COG5 |
| Dystroglycanopathy, Type C, 5 | diseases, Genetic diseases | |
| Cutis Laxa | Neuronal diseases, Eye diseases, Bone | ATP6V0A2 |
| | diseases, Cardiovascular diseases, | |
| | Gastrointestinal diseases, Skin diseases, | |
| | Nephrological diseases, Genetic diseases, | |
| | Metabolic diseases, Rare diseases, Fetal | |
| | diseases | |
| Congenital Dyserythropoietic | Blood diseases, Genetic diseases, Metabolic | SEC23B |
| Anemia | diseases, Rare diseases | |
| Clubfoot | Bone diseases, Genetic diseases, Rare | CHST14 |
| | diseases, Fetal diseases | |
| Osteopetrosis | Eye diseases, Bone diseases, Blood diseases, | ATP6V0A2 |
| | Genetic diseases, Rare diseases, Fetal | |
| | diseases | |
| Cerebrocostomandibular-Like | Rare diseases | COG1 |
| Syndrome | | |
| Hyperphosphatemia | | GALNT3 |
| Hyperostosis | Bone diseases | GALNT3 |
| Irregular Astigmatism | | CHST6 |
| Testicular Microlithiasis | Reproductive diseases, Genetic diseases | GALNT3 |
| Astigmatism | Eye diseases | CHST6 |
| Extratemporal Epilepsy | | EXT1 |
| Isolated Hyperckemia | Genetic diseases | FKRP |
| Laryngomalacia | Respiratory diseases, Oral diseases, Rare | POMGNT1 |
| | diseases, Fetal diseases | |
| Enophthalmos | Eye diseases | CHST3 |
| Calcinosis | | GALNT3 |
| Autosomal Recessive Disease | | MGAT2 |
| Neuromuscular Disease | Neuronal diseases, Nephrological diseases | FKRP |
| Sialuria | Genetic diseases, Metabolic diseases, Rare | SLC35A1 |
| | diseases | |
| Gastroesophageal Junction | Cardiovascular diseases, Gastrointestinal | EXT1 |
| Adenocarcinoma | diseases | |
| Larsen Syndrome | Bone diseases, Genetic diseases, Rare | CHST3 |
| | diseases, Fetal diseases | |
| Hypohidrosis | | COG6 |
| Congenital Contractures | Rare diseases | CHST14 |
| Mucopolysaccharidoses | | CHST14 |
| Corneal Disease | Eye diseases | CHST6 |
| Siderosis | Respiratory diseases, Rare diseases | SEC23B |
| Oculocerebrorenal Syndrome | Neuronal diseases, Eye diseases, | COG4 |
| | Nephrological diseases, Metabolic diseases, | |
| | Rare diseases, Fetal diseases | |
| Hutchinson-Gilford Progeria | Genetic diseases | B4GALT1 |
| Pulmonary Tuberculosis | Respiratory diseases | CHST14, |
| | | LARGE |
| Agammaglobulinemia | Blood diseases, Immune diseases, Genetic | LARGE |
| | diseases, Rare diseases, Cancer diseases | |
| Dilated Cardiomyopathy | Cardiovascular diseases, Genetic diseases, | FKRP |
| | Rare diseases | |
| Huntington Disease | Neuronal diseases, Genetic diseases, Rare | SLC35A1, |
| | diseases | SEC23A |
| Systemic Lupus Erythematosus | Bone diseases, Skin diseases, Genetic | LARGE |
| | diseases, Rare diseases | |
| Becker Muscular Dystrophy | Neuronal diseases, Muscle diseases, Genetic | FKRP |
| | diseases, Rare diseases | |
| Systemic Onset Juvenile | Bone diseases, Respiratory diseases, Rare | COG5 |
| Idiopathic Arthritis | diseases | |
| Renal Oncocytoma | Nephrological diseases, Genetic diseases, | GALNT3 |
| | Rare diseases, Cancer diseases | |
| Alcoholic Hepatitis | Gastrointestinal diseases, Liver diseases | B4GALT1 |
| Hereditary Spastic Paraplegia | Neuronal diseases, Mental diseases, Eye | SEC23A |
| | diseases, Bone diseases, Gastrointestinal | |
| | diseases, Genetic diseases, Metabolic | |
| | diseases, Rare diseases | |
| Cervical Cancer, Somatic | Reproductive diseases, Genetic diseases, | SEC23A |
| | Cancer diseases | |
| Respiratory Syncytial Virus | Respiratory diseases | LARGE |
| Infectious Disease | | |

While providing guidance with respect to some medical conditions, the below description is not aimed at being limiting but rather shed light on the rational of treatment according to the present teachings.

1. Diseases associated with immunoglobulin secretion, e.g., heavy chain e.g., γ and μ heavy chain diseases. Examples of such diseases include γ-HCD and μ-HCD.

2. Plasma cell dyscrasias, B cell malignancies, (myeloma e.g., multiple myeloma, chronic lymphocytic leukemia (CLL). Although secretion of monoclonal immunoglobulins is a typical feature of plasma cell dyscrasias, it can also be detected in other B cell malignancies including CLL. Serum Free Light Chains (FLC) have prognostic significance in monoclonal gammopathy of undetermined significance, solitary plasmocytoma of bone, smouldering myeloma, multiple myeloma, Waldenstroms macroglobulinaemia and AL amyloidosis. Multiple myeloma is a cancer of antibody-secreting plasma cells, wherein aberrant antibodies are secreted in great volume into the blood stream, interfering with the normal titer of blood-borne antibodies and enhancing the risk of kidney failure. The Golgi apparatus in multiple myeloma cells endures heavy protein load and thus constitutes a lucrative target for anti-cancer therapeutics. By specifically targeting the GQC pathway, it is possible to stress cells to the point where secretory, cancerous cells would be effected to a far greater extent than healthy cells, tipping the balance of protein stress in favor of cell death specifically in these cells.

3. Viral infection—The propagation of viruses such as Influenza and HIV Herpes virus, Poxvirus, Falvivirus, Togavirus, Coronavirus, Hepatitis D virus and Rhabdovirus relies on mammalian cells synthesizing and exporting glycoproteins that imbed into the viral particles. By affecting the GAD pathway, it is possible to target viral glycoproteins to degradation, effectively inhibiting the maturation of viral particles from infected cells.

4. Certain sugars are added to glycoproteins specifically in the Golgi. These include Galactose, Fucose and Sialic acid. Non-sialylated glycoproteins are quickly removed from the bloodstream, thus it is contemplated that this modification undergoes QC. The addition of sialic acid has been shown to be crucial for many processes:
Most, if not all, proteins secreted to the bloodstream by the liver are sialylated and their functions have been shown to be linked to the presence of this glyco-modification.
Sialic acid, among other glycans, has been suggested to play a role in fertilization and embryogenesis.
Sialic acid serves as a receptor molecule for Influenza hemagglutinin, allowing specificity of infection for this virus. The addition of sialic acid to plasma membrane proteins occurs in the Golgi prior to localization of these proteins to the plasma membrane. Aberration in sialylation could potentially prevent influenza infection into cells. There are studies on effects of sialylation on dengui and influenza viral infections]. Viral infections and the effects of blood cancers on the sialylation of leukocytes. Increased sialylation of liver cells (cirrhosis) has also been linked to various diseases.

5. In immune response, the production and secretion of antibodies by B-cells is a highly regulated process that ensures the production of valid, functional antibodies and prevents the production of aberrant self-targeting antibodies. Aberrations in the GQC and GAD pathways of secretory B-cells may hold the explanation for the production and secretion of self-targeting antibodies in auto-immune diseases. A link between QC and autoimmunity has recently been hypothesized, but in the context of ER QC. By bolstering the stringency of GQC and GAD, it is possible to inhibit the secretion of auto-immune antibodies.

6. In Alzheimer's disease, the Amyloid beta Precursor Protein (APP) has been shown to accumulate in the Golgi, causing its fragmentation and possibly leading to cell death. Protein accumulation is a hallmark of QC, well established in ER QC. Pathological accumulation however, is an unwanted cellular state that might arise from aberrant QC and degradation machinery. Aberrant protein aggregation and Golgi fragmentation has also been identified in diseases such as ALS, corticobasal degeneration, Alzheimer's disease and Creutzfeldt-Jacob disease.

7. The Golgi apparatus also functions as an intracellular sorting organelle, organizing the trafficking of cargo to their intracellular destination. When this sorting role does not function properly, proteins can accumulate at the Golgi rather than arriving at their destination organelles. The best known sorting signal in the Golgi is the mannose-6-phosphate moiety which targets proteins to the lysosome. Improper trafficking could lead to an accumulation of lysosomal proteins in the Golgi where they would need to be disposed of, causing both a shortage of these proteins in the lysosomes and a GQC load on the Golgi.

8. In many secretory cells, such as hepatocytes, cells of the digestive tract and neurons, proteins are sorted into secretory vesicles at the Golgi. These sorting events could potentially involve GQC as a means to ensure that only functional proteins and cargo are exported from the Golgi in secretory vesicles. Defects in such a proposed pathway could potentially inhibit the function of liver-secreted enzymes, causing accumulation of blood toxins which are normally cleared by such enzymes.

9. The process of inflammation requires inflammatory cytokines to be secreted from specialized cells. When control of this process is hindered, chronic inflammation, an unwanted pathological state, may ensue. The background for these cases may involve aberrations of the GQC and GAD pathways and bolstering these pathways by exogenic means or by specific drug treatments could alleviate the inflammatory cytokine load and cure chronic inflammation.

10. Disproteinemia—typically due to presence of free immunoglobulins in the serum or plasma. Also causes clotting defects due to concurrent thrombocytopenia or to coating of the platelet with the abnormal protein.

The medical conditions listed hereinbelow should follow the treatment themes described hereinabove.

Inflammatory Diseases

Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

According to a specific embodiment, when the disease is an autoimmune disease (e.g., lupus), treatment does not comprise co-treatment of monensin with a nucleic acid agent.

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

Congenital Golgi Diseases

Also known as congenital disorder of glycosylation (previously called carbohydrate-deficient glycoprotein syndrome) in which glycosylation of a variety of tissue proteins and/or lipids is deficient or defective. These diseases are often classified to Type I and Type II disorders.

Type I disorders involve disrupted synthesis of the lipid-linked oligosaccharide precursor (LLO) or its transfer to the protein.

Types include:

| Type | OMIM | Gene | Locus |
|---|---|---|---|
| Ia (PMM2-CDG) | 212065 | PMM2 | 16p13.3-p13.2 |
| Ib (MPI-CDG) | 602579 | MPI | 15q22-qter |
| Ic (ALG6-CDG) | 603147 | ALG6 | 1p22.3 |
| Id (ALG3-CDG) | 601110 | ALG3 | 3q27 |
| Ie (DPM1-CDG) | 608799 | DPMI | 20q13.13 |
| If (MPDU1-CDG) | 609180 | MPDU1 | 17p13.1-p12 |
| Ig (ALG12-CDG) | 607143 | ALG12 | 22q13.33 |
| Ih (ALG8-CDG) | 608104 | ALG8 | 11pter-p15.5 |
| Ii (ALG2-CDG) | 607906 | ALG2 | 9q22 |
| Ij (DPAGT1-CDG) | 608093 | DPAGT1 | 11q23.3 |
| Ik(ALGl-CDG) | 608540 | ALG1 | 16p13.3 |
| 1L (ALG9-CDG) | 608776 | ALG9 | 11q23 |
| Im (DOLK-CDG) | 610768 | DOLK | 9q34.11 |
| In (RFT1-CDG) | 612015 | RFT1 | 3p21.1 |
| Io (DPM3-CDG) | 612937 | DPM3 | 1q12-q21 |
| Ip (ALG11-CDG) | 613661 | ALG11 | 13q14.3 |
| Iq (SRD5A3-CDG) | 612379 | SRD5A3 | 4q12 |
| Ir (DDOST-CDG) | 614507 | DDOST | 1p36.12 |
| DPM2-CDG | n/a | DPM2 | 9q34.13 |
| TUSC3-CDG | 611093 | TUSC3 | 8p22 |
| MAGT1-CDG | 300716 | MAGT1 | X21.1 |
| DHDDS-CDG | 613861 | DHDDS | 1p36.11 |
| I/IIx | 212067 | n/a | n/a |

Type II disorders involve malfunctioning trimming/processing of the protein-bound oligosaccharide chain.
   Types include:

| Type | OMIM | Gene | Locus |
|---|---|---|---|
| IIa (MGAT2-CDG) | 212066 | MGAT2 | 14q21 |
| IIb (GCS1-CDG) | 606056 | GCS1 | 2p13-p12 |
| IIc (SLC335C1-CDG; Leukocyte | 266265 | SLC35C1 | 11p11.2 |
| adhesion deficiency II)) | | | |
| IId (B4GALT1-CDG) | 607091 | B4GALT1 | 9p13 |
| IIe (COG7-CDG) | 608779 | COG7 | 16p |
| IIf (SLC35A1-CDG) | 603585 | SLC35A1 | 6q15 |
| IIg (COG1-CDG) | 611209 | COG1 | 17q25.1 |
| IIh (COG8-CDG) | 611182 | COG8 | 16q22.1 |
| IIi (COG5-CDG) | 613612 | COG5 | 7q31 |
| IIj (COG4-CDG) | 613489 | COG4 | 16q22.1 |
| IIL (COG6-CDG) | n/a | COG6 | 13q14.11 |
| ATP6V0A2-CDG (autosomal | 219200 | ATP6V0A2 | 12q24.31 |
| recessive cutis laxa type 2a | | | |
| (ARCL-2A)) | | | |
| MAN1B1-CDG (Mental retardation, | 614202 | MAN1B1 | 9q34.3 |
| autosomal recessive 15) | | | |
| ST3GAL3-CDG (Mental retardation, | 611090 | ST3GAL3 | 1p34.1 |
| autosomal recessive 12) | | | |

Disorders with Deficient α-Dystroglycan O-Mannosylation.

Mutations in several genes have been associated with the traditional clinical syndromes, termed muscular dystrophy-dystroglycanopathies (MDDG). A new nomenclature based on clinical severity and genetic cause was recently proposed by OMIM. The severity classifications are A (severe), B (intermediate), and C (mild). The subtypes are numbered one to six according to the genetic cause, in the following order: (1) POMT1, (2) POMT2, (3) POMGNT1, (4) FKTN, (5) FKRP, and (6) LARGE.

Most common severe types include:

| Name | OMIM | Gene | Locus |
|---|---|---|---|
| POMT1-CDG (MDDGA1; | 236670 | POMT1 | 9q34.13 |
| Walker-Warburg syndrome) | | | |
| POMT2-CDG (MDDGA2; | 613150 | POMT2 | 14q24.3 |
| Walker-Warburg syndrome) | | | |
| POMGNT1-CDG (MDDGA3; | 253280 | POMGNT1 | 1p34.1 |
| muscle-eye-brain) | | | |
| FKTN-CDG (MDDGA4; Fukuyama | 253800 | FKTN | 9q31.2 |
| congenital muscular dystrophy) | | | |
| FKRP-CDG (MDDGB5; MDC1C) | 606612 | FKRP | 19q13.32 |
| LARGE-CDG (MDDGB6; MDC1D) | 608840 | LARGE | 22q12.3 |

Neurodegenerative Diseases

Exemplary neurodegenerative diseases include, but are not limited to Huntington's Disease (HD), Alzheimer's Disease (AD), aging, retinal degeneration and stroke.

Additional neurodegenerative diseases include Parkinson's disease, Multiple Sclerosis, ALS, multi-system atrophy, progressive supranuclear palsy, fronto-temporal dementia with Parkinsonism linked to chromosome 17 and Pick's disease.

Oxidative Stress Conditions

The phrase “oxidative stress conditions” as used herein, refers to conditions that elevate the level of reactive oxidative species (ROS) beyond the normal level. As mentioned this may result from a lack of antioxidants or from an over abundance free radicals. Exemplary ROS conditions include, but are not limited to 6-hydroxydopamine toxicity, hydrogen peroxide toxicity, UV radiation and dopamine toxicity.

The agent of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agent) effective to prevent, alleviate or ameliorate symptoms of a disorder (e.g., viral disease, cancer, autoimmune, inflammatory, neurodegenerative disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Diagnostics

According to an aspect of the invention there is provided a method of diagnosing a medical condition. The method comprising analyzing activity or expression of the GQC machinery in a subject in need thereof, wherein an aberrant activity or expression of the GQC in the subject is indicative of a medical condition.

As used herein “aberrant” refers to a deviation from the activity or expression of a component of GQC machinery (e.g., listed in FIG. 1C) as compared to same in a normal cell under identical assay conditions.

Methods of analyzing expression and activities of mRNA, proteins are well known in the art. Some are listed above.

Alternatively aberrant GQC can also be detected at the DNA level.

Once aberrant GQC is detected the subject may be directed to further medical examination as well as treatment modalities e.g., as described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Antibodies

Mouse anti (β-COP, Rabbit anti PSMD6, Mouse anti HA (Sigma), Rabbit anti Giantin, Rabbit anti TGN46, Mouse anti P97/VCP (Abcam), Mouse anti Lamin A+C, Mouse anti importinβ, Mouse anti mitochondria (Abcam), Mouse anti Grp78 (Bip), Mouse anti VDAC1, Mouse anti GAPDH, Rabbit anti Hsp90 (Abcam), Mouse anti polyubiquitin (Enzo), Mouse anti 58K-Golgi protein (Abcam), Rabbit anti Grp94, Rabbit anti K-48 linked polyubiquitin (Abcam), Rabbit anti GFP (Abcam), Rabbit anti Calnexin (Cell signaling) Rabbit anti PSMD11 and Rabbit anti PSMD14 were kind gifts from. Mouse anti alpha-4 (Santa Cruz), Mouse anti alpha-6, produced from hybridoma was a kind gift from Tanaka, K G, Rabbit anti Lamp 1 was a kind gift from Zvulun Elazar. Anti-mouse CD138 and CD19 tagged with APC and PE respectively (BD biosciences). Goat anti Mouse 488, Goat anti Rabbit 549, Goat anti mouse 647, Goat anti Rabbit 647 (Invitrogen) Goat anti mouse HRP, Goat anti Rabbit HRP (Jackson labs).

Cell Culture and Drug Treatments

HEK 293, IMR-90 and HeLa cells were grown in DMEM supplemented with 10% fetal bovine serum, 1% Penicillin/streptomycin and L-glutamine (2 mmole/liter) (Biological industries) at 37° C. with 5% CO2. Drugs were introduced to cells for 2 hrs at varying concentrations. Tunicamycin (Sigma) was given at 5 μg/ml. Treatment with the glycosylation inhibitor tunicamycin is known to cause accumulation of unfolded glycoproteins in the ER which is followed by poly- and degradation of these aberrant proteins. In the Golgi, tunicamycin has been shown to inhibit the cross-membrane trafficking of UDP-Galactose (Yusuf et al., 1983; Yusuf et al., 1984), thus inhibiting the addition of this sugar moiety to maturing glycoproteins. Monensin (Enco scientific services) was given at 2 mM and MG-132 (Biotest) at 40 μM. Monensin is an antibiotic, commercially known as Golgi-stop. This drug inhibits the transport of proteins between the medial and trans-Golgi stacks thus also inhibiting phosphorylation, addition of galactose to glycoproteins and sulfation that occur in the trans-Golgi (Griffiths et al., 1983; Rosa et al., 1992). Cell death assessment was done by trypan blue staining and counted with Countess II™ automated cell counter (Thermo Fisher). Proliferation of mammalian cells was measured by XTT assay.

### Sucrose Cushions and Golgi Fractionation

Cells were grown to confluence in 4-15 cm plates before being scraped and homogenized in swelling buffer (Merbl and Kirschner, 2014) using 20 strokes in a kontess dounce homogenizer. Homogenate was then centrifuged at 1,000 G for 10 min in order to pellet nuclei and debris, supernatant was then centrifuged at 100,000 G for 1 hr yielding a cytosolic supernatant and a membranous pellet. This pellet was solubilized in 0.5M sucrose and overlaid onto 0.86M sucrose to form a sucrose cushion. The sucrose cushion was centrifuged at 100,000 G for 1 hr, leaving Golgi fractions in the upper portion of the cushion, ER and lysosomal fractions in the lower part and a membranous fraction in the interface of these two concentrations. Purity of fractions is validated by SDS-PAGE.

### Immunofluorescence Microscopy

HeLa cells, grown on 96-well ‘cell carrier’ plates (Perkin Elmer) were fixed in 4% paraformaldehyde (Electron microscopy sciences) and permeabilized in 0.5% triton (sigma)—PBS (biological industries). Primary antibodies were introduced for 1 hr and secondary antibodies for 30 min, both in PBS-2% BSA. Hoechst staining (Sigma) was done per product protocol. Images were acquired using the ‘Operetta’ high content screening microscope at ×40 magnification and analyzed by ‘Harmony’ software (Perkin Elmer).

### In Vitro

Purified fractions were incubated with energy mix and recombinant HA tagged ubiquitin as previously described (Merbl et al., 2013) and either immediately boiled in Laemmli buffer and β-mercaptoethanol or allowed to incubate at room temperature for 30 to 60 minutes. All samples were then analyzed by SDS-PAGE using mouse anti HA primary and goat anti mouse—HRP secondary antibodies. Western blots were quantified using Fiji software.

Proteasome Cleavage Reporter Assay

Golgi or ER fractions from drug/siRNA treated HEK293 cells were incubated with suc-LLVY-AMC (Biotest) as per protocol and fluorescence levels were measured over time using a Tecan M200 plate reader (Ex: 360 nm, Em: 460 nm).

### SiRNA Transfection and RT-PCR Analysis

ON-TARGET plus smart-pool siRNA for different targets (Dharmacon) were inserted by lipofectamine 2000 transfection (Invitrogen). mRNA levels were ascertained by real time quantitative PCR using syber-green (Kapa Biosystems) using primers as outlined:

Bip
(SEQ ID NO: 1)
TGTTCAACCAATTATCAGCAAACTC
(SEQ ID NO: 2)
TTCTGCTGTATCCTCTTCACCAGT
CHOP
(SEQ ID NO: 3)
AGAACCAGGAAACGGAAACAGA
(SEQ ID NO: 4)
TCTCCTTCATGCGCTGCTTT
XBP1s
(SEQ ID NO: 5)
CTGAGTCCGAATCAGGTGCAG
(SEQ ID NO: 6)
ATCCATGGGGAGATGTTCTGG
PSMD6
(SEQ ID NO: 7)
AGCCCTAGTAGAGGTTGGCA
(SEQ ID NO: 8)
AGGAGCCATGTAGGAAGGC,
SLC35A1
(SEQ ID NO: 9)
CTGTGTGCTGGAGTTACGCT
(SEQ ID NO: 10)
TACTCCTGCAAATCCTGAGC
GAPDH
(SEQ ID NO: 11)
CAACGGATTTGGTCGTATTG
(SEQ ID NO: 12)
GATGACAAGCTTCCCGTTCT

SEM/TEM and Immuno-Gold Staining

Purified Golgi fractions collected from HEK 293 cells were fixed in 4% paraformaldehyde, 2% glutaraldehyde, in cocodylate buffer containing 5 mM CaCl2 pH=7.4. After fixation, the samples were washed 3-4 times in 0.1M sodium cacodylate buffer (5 min each) in order to remove all the aldehyde excess. The samples were then plated over-night at 40 C on silicon wafer coated with poly-L-lysine 1 mg/ml. The samples were then incubated for 1 hour in 1% osmium tetroxide in 0.1M Na cacodylate buffer, washed in cacodylate buffer and then dehydrated in ethanol before being dried in a critical point dryer (CPD) and mounted onto stabs and coated with carbon at 20 nm thickness.

In-Vivo Assays

5TMM mice, a breed of C57BL/KalwRij mice that are sensitive to MM, were injected with 5TGM1 murine MM cell line and blood levels of IgG2B were measured periodically over 32 days by ELISA. Mice were split into 2 groups, the control group received 0.35% ethanol in drinking water while the test group received 80 μM monensin (initially solubilized in 70% ethanol) in drinking water. Mice were sacrificed after 5 days of treatment. Spleens and bone marrow were harvested, homogenized and analyzed by FACS.

### Example 1

### Bioinformatic Analysis Reveals Prevalent Protein Ubiquitylation in Golgi-Associated Proteins

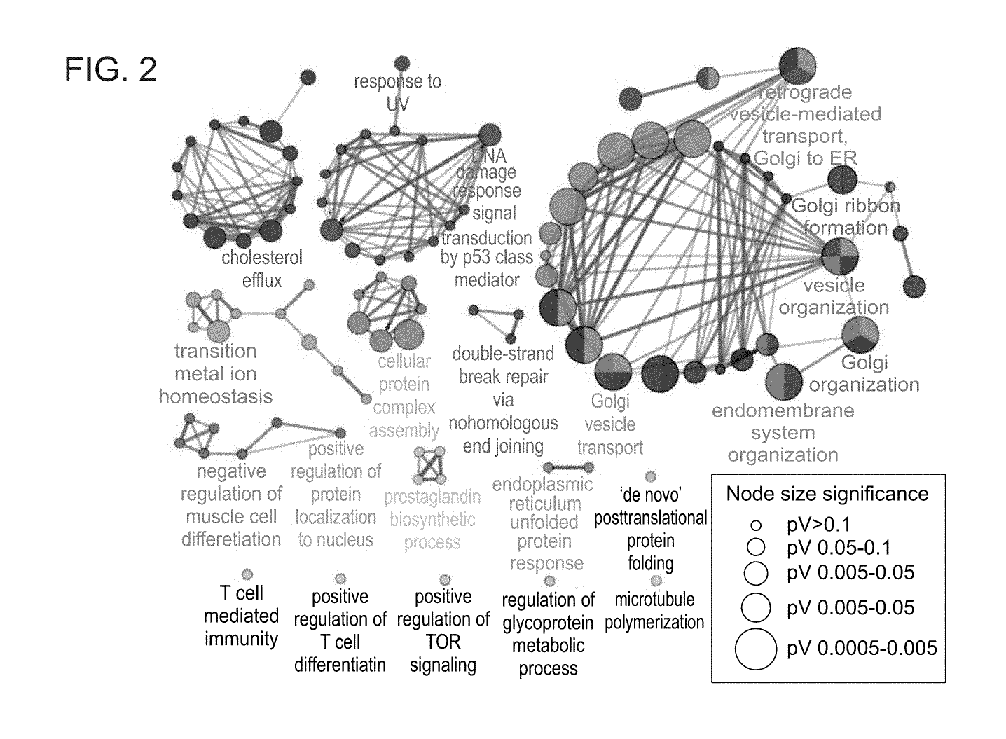

Various post translational modifications, have been shown to occur in the Golgi apparatus, these include protein phosphorylation (24-26), acetylation (27-29), glycosylation, modification with ISG15 (30) ubiquitylation (18, 19, 31) and others. Specifically, the roles of protein ubiquitylation in the Golgi have been under-investigated, as very few ubiquitin machinery proteins have been identified in the Golgi. In order to better understand the frequencies of PTMs in the Golgi apparatus, a bioinformatics analysis was performed, comparing the PTM landscape of Golgi proteins to those of the ER. Protein localization data published in the human protein atlas database (32) was used to classify proteins as being localized to the Golgi, the ER or both and supplemented this localization data with the PTM database PTMcode2 (33). Quantifying the PTMs of various proteins and their localizations shows that the frequency of many PTMs in the Golgi is comparable to that of the ER (FIG. 1A). Specifically, phosphorylation, ubiquitylation and acetylation occur in highly similar frequencies in these two organelles. These data suggest that ubiquitylation in the Golgi occurs in many more proteins than previously appreciated and could play a far greater role in the Golgi apparatus than acknowledged. In order to overview the roles and molecular functions of Golgi localized ubiquitylated proteins, the PANTHER classification system (34) was used to obtain GO-term classification for the subset of proteins (FIG. 1B). From this analysis, it is clear that Golgi localized proteins that undergo ubiquitylation are involved in a variety of molecular functions in the Golgi. Using Cytoscape software (35) with the ClueGo plugin (36), the interaction network of ubiquitylated proteins was visualized in the Golgi (FIG. 2). The resulting graphic visualizes the various pathways in which ubiquitylated proteins are involved in the Golgi, some of which were expected such as Golgi organization and Golgi vesicle transport while some were surprising such as cholesterol efflux and response to UV. The scope and convolution of this network helps appreciating that indeed, ubiquitylation has a more important role in the Golgi apparatus than previously known.

The Golgi apparatus contains proteins with ubiquitin associated domains, as inferred from the data gathered from the human protein atlas, cross referenced with UniProt annotations (FIG. 1C). These include ubiquitin E3 ligases, DUBs, PHD containing proteins, various ubiquitin like containing proteins and proteasomal subunits.

Taken together, these data show that ubiquitylated proteins are involved in many different pathways in the Golgi and that ubiquitylation is a largely overlooked yet highly influential modification that occurs in the Golgi to a greater extent than was previously known.

### Example 2

### Constitutive and Stress-Induced Ubiquitylation of Proteins Retained in the Golgi

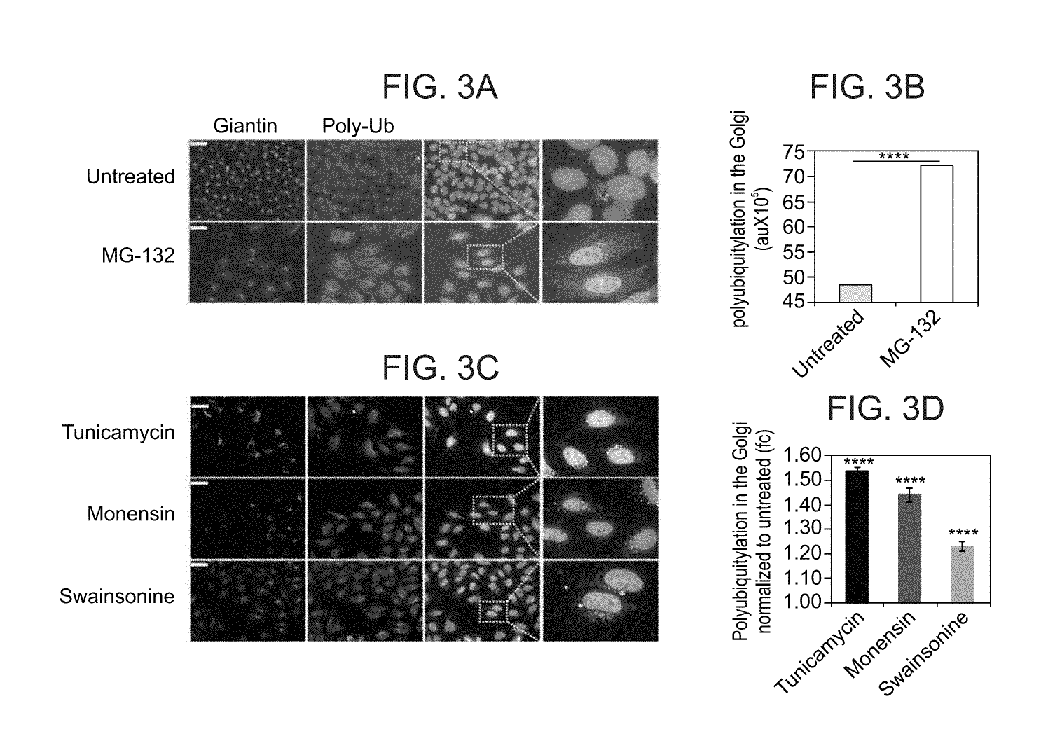

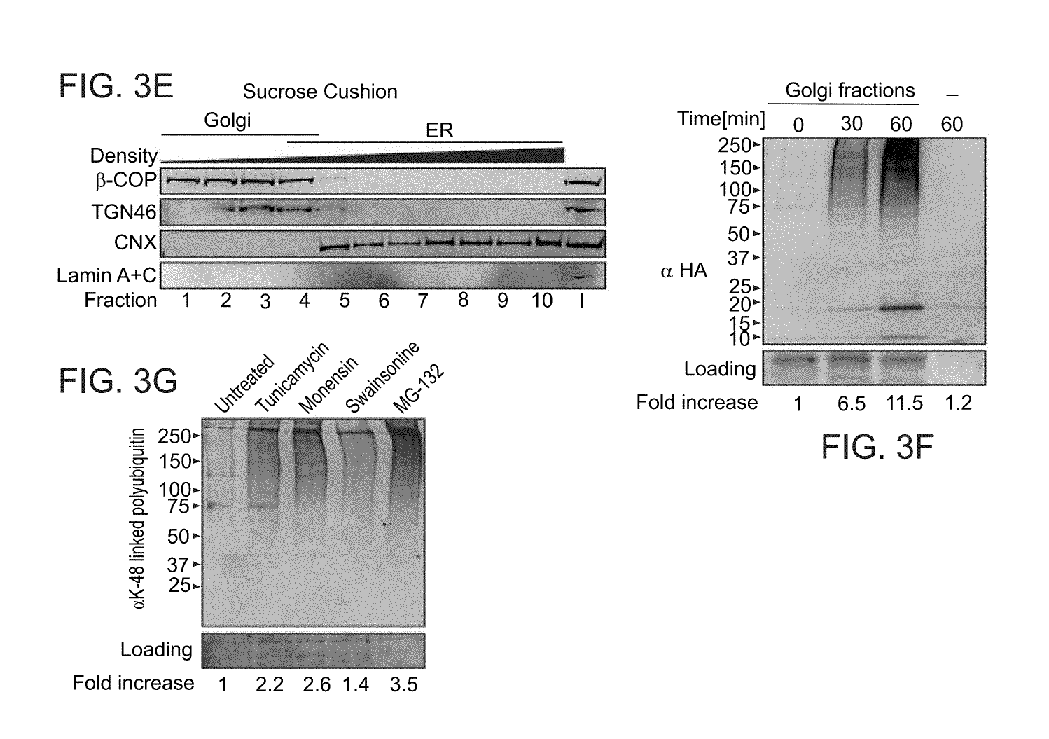

The bioinformatics analysis of the extent of ubiquitylation in the Golgi prompted us to confirm the prediction for Golgi ubiquitylation in live mammalian cells. To this end, a high content screening microscopy system was utilized to assess and quantify the levels of polyubiquitylation in the Golgi apparatus under various stress inducing drug treatments. For this assay, drugs known to induce cell stress by different pathways were selected. Immunofluorescence images of HeLa cells stained for the Golgi marker giantin and poly-ubiquitin show Golgi localized polyubiquitylated puncta, indicating a basal level of protein steady state polyubiquitylation in the Golgi (FIG. 3A). Proteasomal inhibition is known to cause the accumulation of polyubiquitylated proteins that are bound for proteasomal degradation in the cell (37).

Next, the present inventors examined whether or not this accumulation also occurs in the Golgi, following treatment with the proteasomal inhibitor MG-132. As expected, HeLa cells treated with the proteasomal inhibitor MG-132 show a dramatic increase in polyubiquitylated species in the entire cell (FIG. 3A). Under proteasomal inhibition, a large accumulation of Golgi-localized polyubiquitylated proteins was evident (FIG. 3B). This increase in polyubiquitylated proteins in the Golgi points to the possibility that as in the case of ERAD, polyubiquitylated proteins that are bound for degradation accumulate in the Golgi as well as in the ER upon proteasomal inhibition. Treatment with the glycosylation inhibitor tunicamycin is known to cause accumulation of unfolded glycoproteins in the ER which is followed by poly-ubiquitylation and degradation of these aberrant proteins. In the Golgi, tunicamycin has been shown to inhibit the cross-membrane trafficking of UDP-Galactose (38, 39), thus inhibiting the addition of this sugar moiety to maturing glycoproteins. HeLa cells treated with tunicamycin (FIG. 3C) show a significant increase in Golgi-localized polyubiquitin (FIG. 3D), suggesting that tunicamycin causes a higher load of degradation-bound proteins in the Golgi. Monensin is an antibiotic, commercially known as Golgi-stop. This drug inhibits the transport of proteins between the medial and trans-Golgi stacks thus also inhibiting phosphorylation, addition of galactose to glycoproteins and sulfation that occur in the trans-Golgi (40, 41). Treatment of HeLa cells with monensin also caused an increase in Golgi localized poly-ubiquitin, to slightly lower levels than those measured with tunicamycin (FIG. 3D). This increase in Golgi polyubiquitin is indicative of the accumulation of proteins in the medial Golgi, which could be polyubiquitylated prior to degradation. Swainsonine, an inhibitor of Golgi α-mannosidase II (42) inhibits the maturation of glycoproteins and has been shown to cause the accumulation of glycoproteins in mammalian cells (43). Treatment of HeLa cells with swainsonine causes an increase in Golgi localized poly-ubiquitylation, albeit to a lesser extent when compared with tunicamycin and monensin (FIG. 3D). The increase in poly-ubiquitylation in the Golgi apparatus following treatment with these drugs, is indicative of misfolded protein load in this organelle, as is the case in ERAD where an increase in misfolded protein load brings about an increase in protein polyubiquitylation, targeting misfolded proteins to degradation (2, 44). Under proteasomal inhibition, most polyubiquitylated ERAD substrates accumulate in the lumen of the ER as their retrotranslocation to the cytosol in coupled with degradation (2, 45).

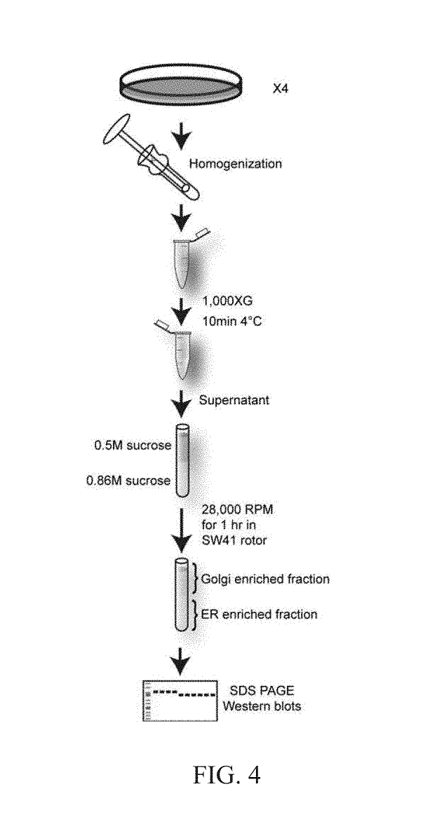

Polyubiquitylation of proteins in the Golgi could occur by either ubiquitylation machinery in the Golgi lumen or, potentially, by cytosolic machinery acting on transmembrane Golgi proteins. In order to address this issue, classical methods were modified for the isolation of Golgi stacks from rat liver (46, 47) in order to allow the purification of intact, functional Golgi's from mammalian cell culture (FIG. 4). Briefly, cells were grown to ˜80% confluence on 15 cm plates and homogenized in 0.5M sucrose using a Dounce homogenizer. Homogenates were centrifuged at 1,000 G to precipitate debris and nuclei and then at 3,000 G. The resulting supernatant was loaded onto a volume of 0.86M sucrose and centrifuged by ultracentrifuge at 28,000 RPM for 1 hour. Then the preparation was ran on fractionated sucrose cushion by SDS-PAGE in order to examine the purity of the Golgi fractions (FIG. 3E). Western blots against Golgi markers (β-COP and TGN46 show Golgi membranes in fractions 1-4, with only the 4^{th} fraction overlapping with ER membranes, visualized by calnexin. Nuclei (lamin A+C) are present in the input and were removed from the homogenate by centrifugation prior to loading on the sucrose cushion. The purification of Golgi fractions allows conducting biochemical experiments on Golgis, isolated from the cellular context. In order to validate the results obtained by immunofluorescence, ubiquitylation activity assays were conducted in purified Golgi fractions. In this assay, recombinant HA-tagged ubiquitin was added to Golgi fractions along with energy mix and incubated for 0, 30 and 60 minutes. Samples were run by SDS-PAGE and western blotted using α-HA antibody. Without incubation, minimal staining could be observed, indicating that no ubiquitylation has occurred. Following 30 minutes of incubation, high molecular weight ubiquitylated proteins appear, indicating that polyubiquitylation occurs in these isolated Golgi fractions without requirement for cytosolic machinery. This polyubiquitylation increased following 60 minutes of incubation and did not occur in the absence of Golgi fractions (FIG. 3F). Polyubiquitin chains can be formed on various lysine residues of ubiquitin, resulting in different linkage-types, not all of which lead to protein degradation. Specifically, K-48 linked polyubiquitin is known to cause proteasomal degradation of proteins and therefore the present inventors tested whether or not the increase in polyubiquitylation indeed potentially leads to protein degradation.

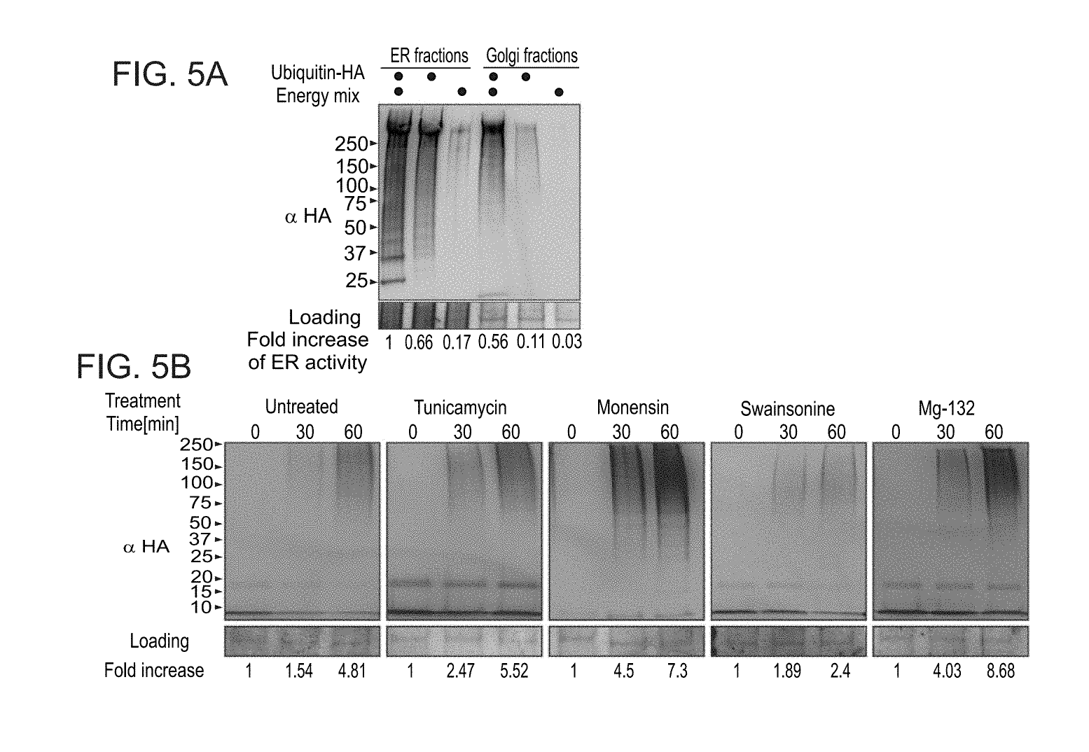

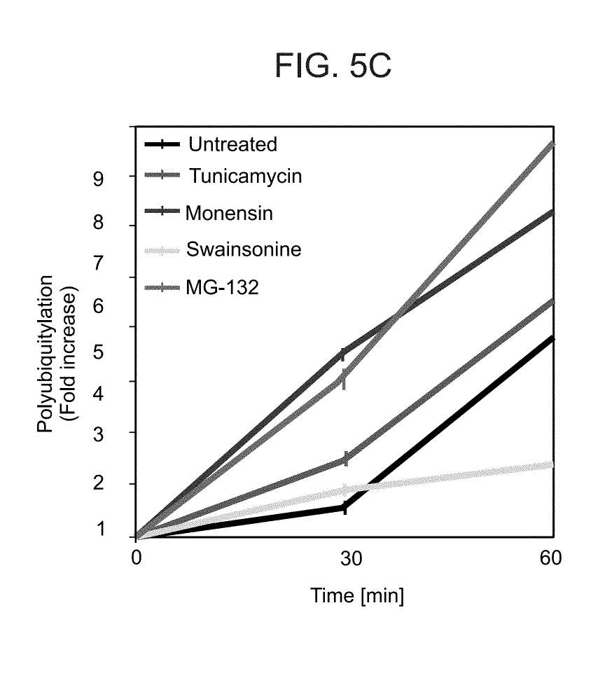

Cells were incubated with the drugs mentioned above, purified Golgi fractions and blotted against K-48 linked polyubiquitin. In accordance to the increase in general polyubiquitin, K-48 linked polyubiquitin specifically increased under proteotoxic stress (FIG. 3G). The Golgi's capacity for ubiquitylation is lower than that of the ER and both fractions were capable of ubiquitylation without the addition of external ATP (FIG. 5A). The effects of proteotoxic drugs on the ubiquitylation capacity of the Golgi (FIGS. 3A-D) are also seen in in-vitro ubiquitylation activity assays (FIG. 5B) with similar results (FIG. 5C). The ability of purified Golgi fractions to produce polyubiquitylated proteins, indicates that the complete ubiquitylation machinery is present in the Golgi itself, without requirement for cytosolic proteins. Moreover, the detection of K-48 linked polyubiquitylated proteins in the Golgi and their increase under proteotoxic stress points to a mechanism for possible degradation of proteins in the Golgi.

### Example 3

### PSMD6, a Regulatory Proteasomal Subunit is Localized to the Golgi and Required for Ubiquitin-Dependent Degradation

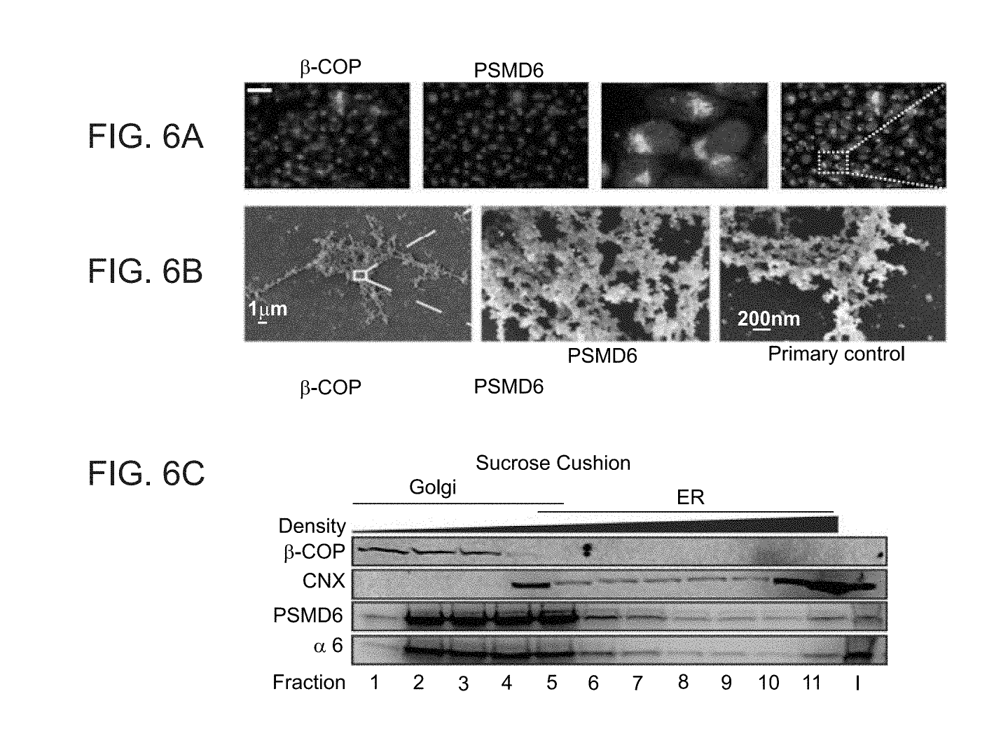

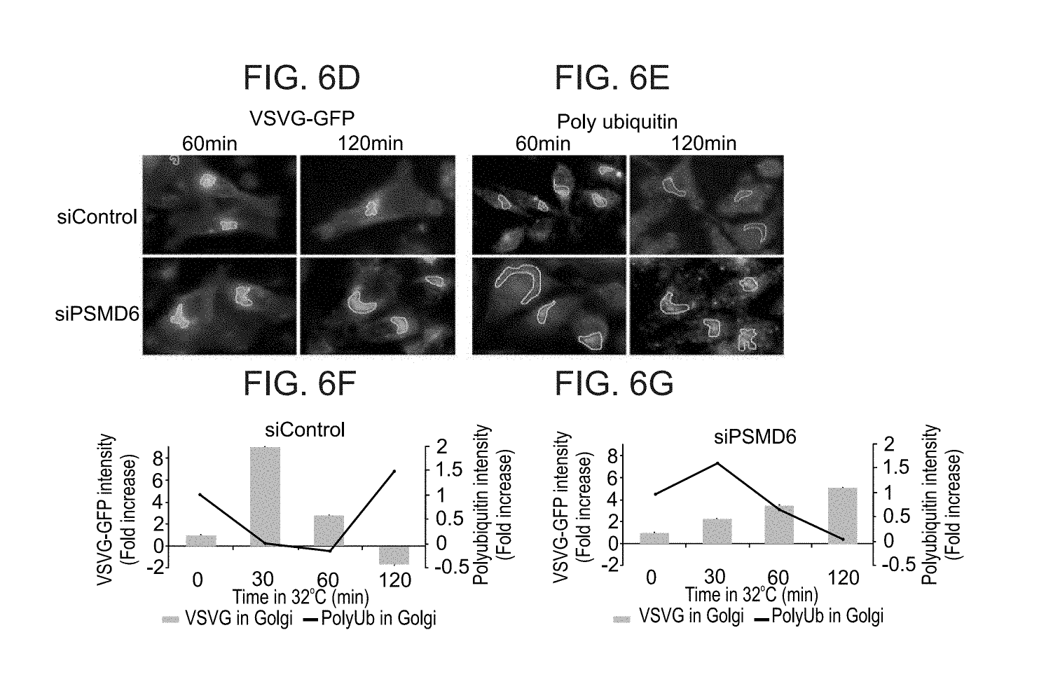

In the above bioinformatic examination of Golgi proteins, based on the human protein atlas, a 19S regulatory non-ATPase subunit of the proteasome, PSMD6, was found to be localized in the Golgi apparatus. Immunofluorescence experiments, co-staining PSMD6 and the Golgi marker β-COP showed colocalization of PSMD6 mainly with the Golgi (FIG. 6A), corroborating the protein atlas database. Taking a high-resolution approach, PSMD6 was visualized using immuno-gold staining of purified Golgis in scanning electron microscopy (SEM). Using SEM allowed detecting PSMD6 (FIG. 6B, yellow dots) on purified Golgis. A primary control, wherein immunogold labeled secondary antibodies were exposed to Golgi fraction without primary antibody incubation, showed a minimal amount of non-specific staining. Seeing as these fractions are purified without the use of membrane-permeabilizing detergents, these SEM images indicate that PSMD6 is bound to the Golgi membrane. Western blots of fractionated cells, blotted against PSMD6, also indicate that this proteasomal subunit is primarily localized to the Golgi and that other proteasomal subunits are also found in the Golgi (FIG. 6C), possibly facilitating proteasomal degradation.

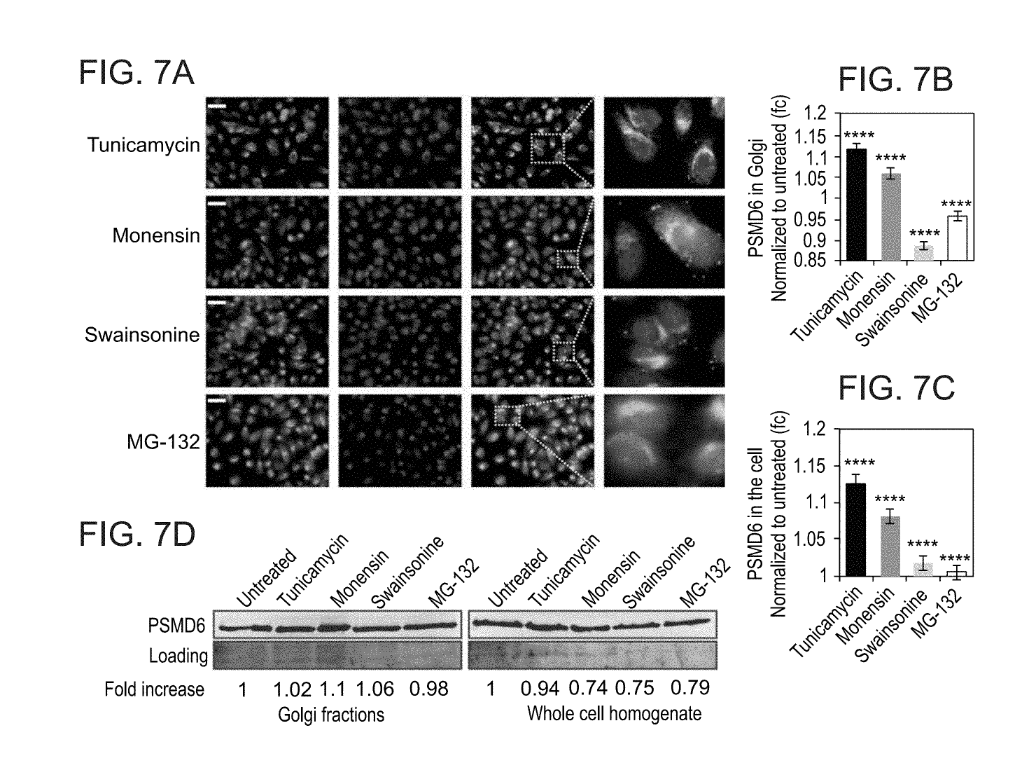

In order to ascertain if PSMD6 levels in the Golgi change in response to proteotoxic stress to mirror the increase in polyubiquitylation, high content microscopy (FIG. 7A) was utilized. Levels of PSMD6 in the Golgi changed to a very minor extent following treatment with proteotoxic stressors (FIG. 7B) and its levels in the entire cell were also stable (FIG. 7C). These results are confirmed by western blot analysis of drug-treated purified Golgi and whole cell homogenate (FIG. 7D).

Under conditions of proteasomal inhibition, ERAD substrates are known to accumulate in the ER (2, 45). The accumulation of misfolded proteins is a hallmark of ER quality control, as the ER can identify misfolded and unfolded proteins in order to prevent their exit, facilitating their degradation. One model substrate, used extensively in the research of both ERAD and the secretory pathway, is the temperature sensitive mutant of the vesicular stomatitis virus glycoprotein which is fused to green fluorescent protein (ts045 VSVG-GFP). When cells are incubated at 40° C., ts045 VSVG-GFP cannot fold properly and is retained in the ER for degradation. However, when cells are moved to the permissive temperature of 32° C., ts045 VSVG-GFP folds properly and exits the ER. Kinetic analyses have shown that the peak of Golgi localization for ts045 VSVG-GFP occurs following 60 minutes from moving to the permissive temperature (48) and that following 120 minutes, most ts045 VSVG-GFP can be found on the plasma membrane.

In order to evaluate the importance of PSMD6 for GQC and GAD, a ts045 VSVG-GFP secretion assay was performed under conditions of PSMD6 knock-down using siRNA in mammalian cells. Cells transfected with control siRNA indeed show VSVG-GFP secretion kinetics that match those expected from the literature (FIGS. 6D, F). After 60 minutes at 32° C., VSVG-GFP levels in the Golgi reach a peak that is diminished after 120 minutes at 32° C. Interestingly, polyubiquitylation levels in the Golgi peak after 120 minutes at 32° C. (FIGS. 6E, F), at which time VSVG levels have decreased. Cells transfected with siRNA targeting PSMD6 show an increase in Golgi VSVG levels, that does not diminish after 120 minutes at 32° C. (FIGS. 6D, G) that is not accompanied by an increase in Golgi localized polyubiquitylation (FIGS. 3E, G).

### Example 4

### Enhanced Golgi-Associated Degradation in Response to Proteotoxic Stress Reveals a Role for GQC in Proteostasis Regulation

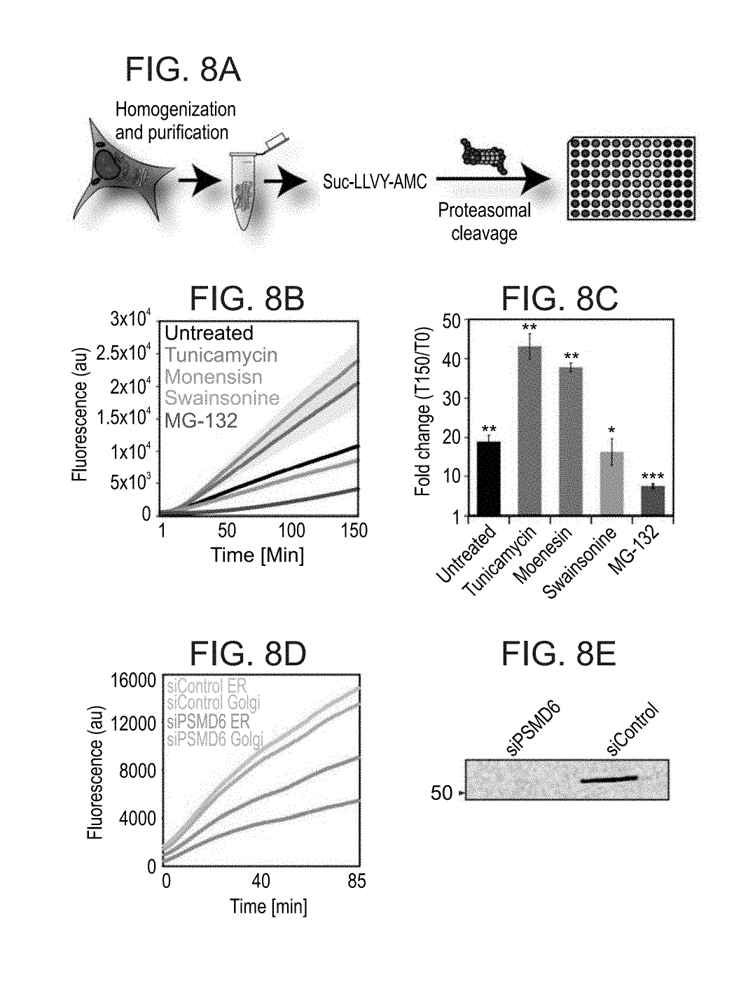

Quality control of protein substrates requires that unfolded and misfolded proteins be identified, ubiquitylated and finally, degraded. Following the above results, showing K-48 linked polyubiquitylation occurring in the Golgi apparatus and the presence of PSMD6, the final step in quality control was examined i.e., degradation. In order to assess degradation levels in the Golgi, the suc-LLVY-AMC fluorogenic proteasomal substrate assay was utilized, wherein the proteasomal substrate was exposed to Golgi fractions from treated vs. untreated cells. Fluorescence levels were measured over time, indicating the increase in proteasomal cleavage product (FIG. 8A). Measuring the fluorescence levels of the proteasomal cleavage product over a period of 150 minutes revealed that the cleavage product accumulates in Golgi fractions, purified from untreated cells (FIG. 8B). This accumulation indicates that active proteasomal cleavage occurs in Golgi fractions independently of other subcellular organelles. Treatment with the proteotoxic stressors Tunicamycin and monensin caused an increase in Golgi-localized degradation (FIGS. 8B, C), which is consistent with their effect on Golgi protein polyubiquitylation (FIG. 3B). The Golgi mannosidase II inhibitor, Swainsonine, seemed to have a small inhibitory effect on degradation of Golgi proteins. The proteasomal inhibitor MG-132, expectedly, caused a distinct inhibition of proteasomal degradation in Golgi fractions (FIGS. 8B, C) that is consistent with this inhibitor's effect of accumulation of polyubiquitylated substrates in the Golgi (FIG. 3D). In order to assess the involvement of PSMD6 in Golgi associated degradation, cells were transfected with either control siRNA or siRNA targeting PSMD6. The cells were fractionated and subjected to a suc-LLVY-AMC assay for both ER and Golgi fractions from cells treated with either siRNA. Knockdown of PSMD6 caused a stark decrease in the degradation capacity of the Golgi and had an inhibitory effect on ER degradation as well, but to a lesser extent (FIG. 8D). Knockdown was assessed by western blot analysis of whole cell homogenates, showing a great reduction in PSMD6 levels in cells treated with siPSMD6 (FIG. 8E). The results of the proteasomal activity assay point to active proteasomal degradation taking place in the Golgi apparatus. Furthermore, PSMD6 is shown to be a central component of Golgi-associated degradation while also playing a role in ERAD.

### Example 5

### Blocking Protein Progression Through the Golgi Apparatus Causes Cell Death

Accumulation of proteins in cells is highly cytotoxic. This cytotoxicity is potentially higher in highly secretory cells, wherein a large mass of proteins is constantly moving through the Golgi apparatus. The present results show that blocking intra-Golgi trafficking by treatment with monensin caused an increase in Golgi-localized polyubiquitylation and degradation. The present inventors examined the long-term effect of Golgi traffic inhibition on secretory cells. Four human cell lines were selected, two that are non-secretory (HEK293 and HeLa) and two secretory cell lines (HepG2 and RPMI 8226) and assessed their live/dead ratios over 2 days of treatment with monensin.

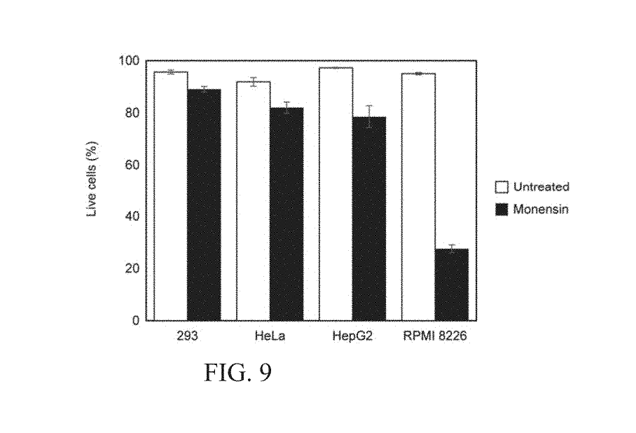

While monensin treatment had little effect on non-secretory cells, secretory HepG2 cells were more susceptible to cell death. This effect was greatest in RPMI 8226 cells, a multiple myeloma (MM) cell line with a heavy secretory load (FIG. 9).

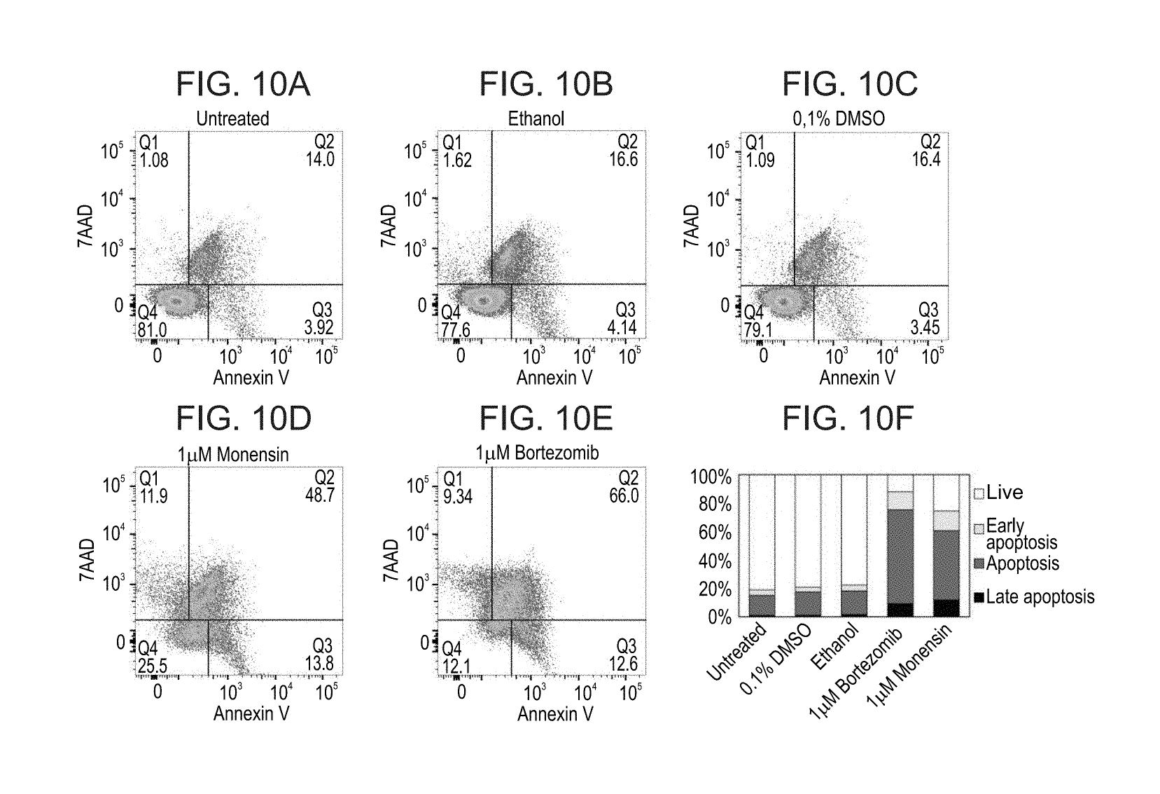

These results were also reproduced in murine 5TGM1 MM cells in FACS experiments using the apoptosis markers annexin V and 7AAD. Untreated cells show an expected low level of cell death, as in controls for DMSO, in which bortezomib is diluted and ethanol, in which monensin is diluted (FIGS. 10A-C). Following only 12 hours of monensin treatment at half the recommended concentration (i.e., 1 μM), apoptotic cell death in 5TGM1 cells was comparable to that found in bortezomib treated cells (FIGS. 10D-E). The similar cell death suggests monensin as a drug of similar treatment potential as bortezomib, for multiple myeloma (FIG. 10F).

### Example 6

### In Vivo Treatment of MM Mice with Monensin

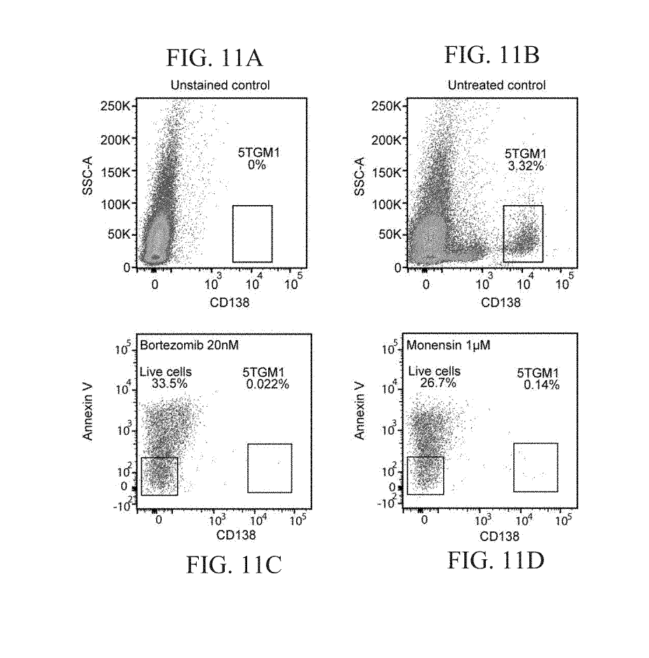

The effect of monensin was tested in MM mice in vivo. To this end bone marrow cells were flushed from C57BL/KalwRij mice injected with 5TGM1 cells. Cells harvested from these mice included MM cells and others, found normally in mouse bone marrow. MM cells were specifically stained with anti CD-138 antibody and gauged apoptosis with Annexin V. Staining control shows the specificity of CD-138 and that the MM cells constitute ˜3.3% of total harvested cells (FIGS. 11A-B). Treatment with bortezomib for 24 hours caused massive cell death of CD-138 positive MM cells and a lesser extent of death in non CD-138 cells (FIG. 11C). The effect of monensin on these cells was comparable to that of bortezomib, affecting mainly the CD-138 positive MM cells (FIG. 11D).

### Example 7

### Combinatorial Treatment for Facilitating Cell Death In Vitro

Monensin effectively inhibits intra-Golgi trafficking in cells and is also known as ‘Golgi-stop’. The above experiments investigating the effect of monensin on cell death in MM stemmed from the understanding that in such high-yield secretory cells, the perturbation of intra-Golgi trafficking would be catastrophic. The proteasomal subunit PSMD6 and the ubiquitin E3 ligase HACE1 are indicated as components of a novel Golgi Apparatus-Related Degradation (GARD) pathway. Following the results with monensin treatments (Examples 5-6), the present inventors wished to examine whether siRNA-mediated downregulation of PSMD6 may sensitize HeLa cells to bortezomib and monensin, by perturbing (and lowering) the Golgi Quality control mechanisms in these cells.

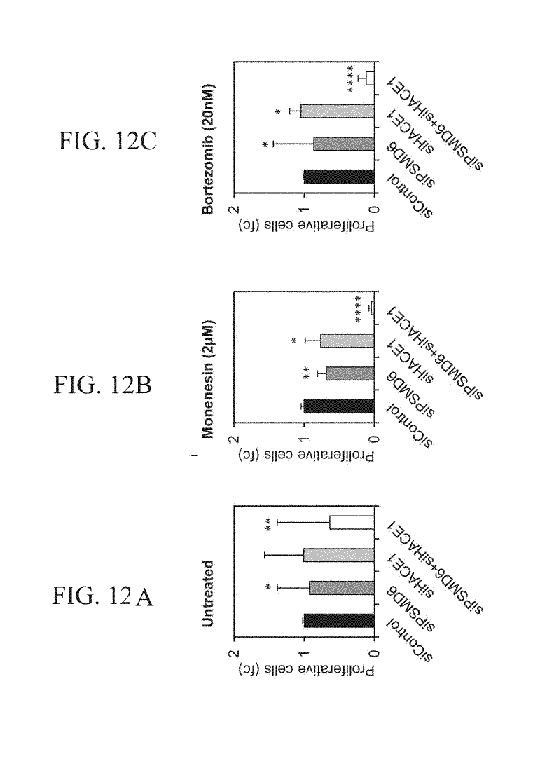

As depicted, individual knockdowns of PSMD6 or HACE1 showed little effect on monensin treated HeLa cells and virtually no effect on bortezomib treated cells (FIGS. 12A-C). However, knockdown of both PSMD6 and Hace1 produced a synergistic effect, effectively sensitizing HeLa cells to both monensin and bortezomib.

These results suggest monensin as a novel anti-cancer drug candidate in the treatment of multiple myeloma and shed new light on the mechanism of action of bortezomib, which is currently not known. Sensitization of bortezomib-resistant MM cells as well as the administration of monensin could potentially offer new hope for treating and possibly curing multiple myeloma. The siRNA sequences are commercially available and consist of a “SMART POOL” of 4 siRNA sequences per gene, from Dharmacon.

### Example 8

### Highly Secretory Multiple Myeloma Cells are Sensitive to Inhibition of Intra-Golgi Trafficking

The physiological role for GARD in the context of multiple myeloma (MM) was examined as cells of MM provide a physiological system for both high glycoprotein production (e.g. antibodies) and secretion. It was found that MM cells are particularly sensitive to monensin-induced cell death compared to other cancer cell lines (FIG. 13A) and that 3 days of treatment killed 99% of MM cells (FIG. 13B). However, even 2 hours of monensin treatment were sufficient for induction of K48 linked polyubiquitination in the Golgi of RPMI 8226 cells (FIG. 13C). This difference may be attributed to the enhanced secretory load in MM cells, which would make them highly reliant on the regulation of Golgi dynamics by processes such as GARD. Thus, artificial deregulation of GARD may simulate such dependency in cells of lesser secretory activity. To allude to this possibility, GARD was inhibited in HeLa cells by co-downregulating the expression of PSMD6 and HACE1, a ubiquitin E3 ligase which was previously shown to ubiqutinate proteins at the Golgi (Zhang, L., Chen, X., Sharma, P., Moon, M., Sheftel, A. D., Dawood, F., Nghiem, M. P., Wu, J., Li, R. K., Gramolini, A. O., et al. (2014). HACE1-dependent protein degradation provides cardiac protection in response to haemodynamic stress. Nature communications 5, 3430) (FIG. 13D). Remarkably, co-depletion of PSMD6 and HACE1 sensitized HeLa cells to monensin and inhibited their proliferation (FIG. 13E). Taken together, our results indicate that perturbation of GARD renders cells sensitive to cell death upon proteotoxic stress. Of note, FIG. 13A shows the response of various cell types to monensin while FIGS. 13D and E show the effect of monensin on transfected HeLa cells only. These are different assays, on different cells showing in FIG. 13E a sensitization to monensin of HeLa cells that are shown in FIG. 13A to be largely insensitive.

### Example 9

### Inhibition of Intra-Golgi Trafficking In-Vivo is a Novel Therapeutic Approach for Multiple Myeloma and Systemic Lupus Erythematosus

In view of the above-results that inhibition of secretion may provide a novel therapeutic opportunity in MM, it was tested whether monensin treatment may inhibit progression of MM disease. To this end, mice were injected with 5TGM1 cells (FIG. 13F) and followed MM progression by monitoring blood levels of IgG2β by ELISA (FIG. 13G). Mice that developed MM were then administered with 80 μM monensin in the drinking water for 5 days. Prominently, monensin-treated mice had significantly lower levels of CD138+, CD19− multiple myeloma cells, both in the spleen (22% to 8%; FIG. 13H) and in bone marrow (BM) (62 to 25%; FIG. 13I). In contrast, CD138−, CD19+ normal B-cell levels were higher than in the control group in both spleen and BM, suggesting that monensin specifically affected multiple myeloma cells. Notably, the spleen size of monensin-treated mice was significantly smaller (˜30%) than that of control mice (FIG. 13J). These findings suggest that monensin treatment may reduce MM manifestations in the in vivo settings.

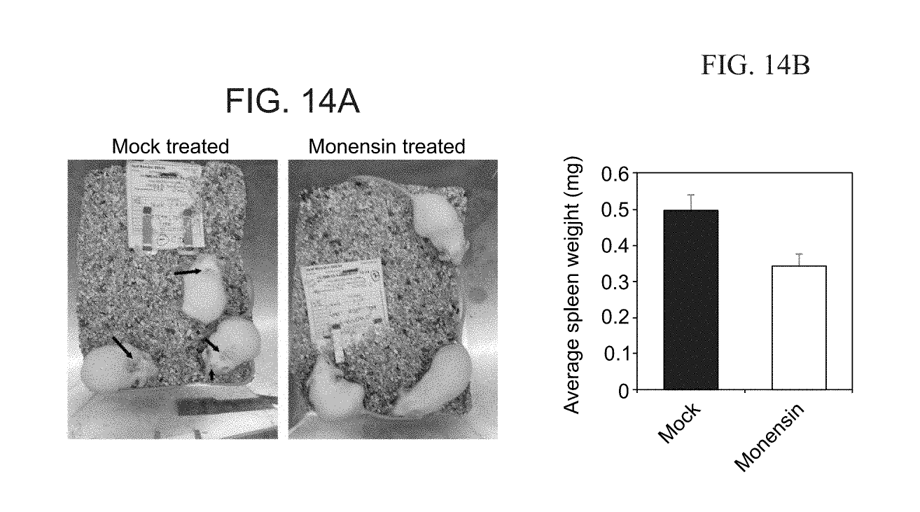

Moreover, in a mouse model of systemic lupus erythematosus, treatment with 80 μM of monensin via drinking water abolished the appearance of skin lesions (FIG. 14A) and significantly reduced splenomegaly of treated mice, as compared to the control group (FIG. 14B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-13. (canceled)

14. A method of treating a pathogenic condition associated with a secreted or membrane presented protein, the method comprising administering to a subject in need thereof an agent that modulates the GQC machinery, thereby treating the pathogenic condition associated with the aberrant protein exocytosis.

15. The method of claim 14, wherein said agent modulates the ubiquitin pathway in the Golgi.

16. The method of claim 15, wherein said agent that modulates the ubiquitin pathway in the Golgi upregulates activity of the ubiquitin pathway in the Golgi.

17. The method of claim 15, wherein said agent that modulates the ubiquitin pathway in the Golgi downregulates activity of the ubiquitin pathway in the Golgi.

18. The method of claim 15, wherein said agent modulates the activity or expression of a component of the ubiquitin pathway in the Golgi.

19. The method of claim 18, wherein said component is selected from the group consisting of an E1 (Ubl), E2, E3, a proteasome subunit, a heat shock protein, a PHD containing protein, a deunbiquitinating enzyme and a regulator of any one of same.

20. The method of claim 18, wherein said component is selected from the group of proteins listed in FIG. 1C.

21. The method of claim 14, wherein said agent modulates protein secretion through the Golgi.

22. The method of claim 21, wherein said agent that modulates protein secretion the Golgi is an inhibitor of protein secretion through the Golgi.

23. The method of claim 14, wherein said agent inhibits COPII anterograde trafficking from endothelial reticulum (ER) to the Golgi.

24. The method of claim 23, wherein said agent is H89.

25. The method of claim 14, wherein said agent alters morphology of the Golgi.

26. The method of claim 25, wherein said agent is megalomicin.

27. The method of claim 14, wherein said agent inhibits glycosylation.

28. The method of claim 27, wherein said agent inhibits sialyltransferase.

29. The method of claim 28, wherein said agent is lythocholyglycine.

30. The method of claim 14, wherein said condition is a pathogenic infection.

31. The method of claim 14, wherein said condition is cancer.

32. The method of claim 31, wherein said cancer is multiple myeloma (MM).

33. The method of claim 14, wherein said condition is an autoimmune disease.

34. The method of claim 33, wherein said autoimmune disease is systemic lupus erythematosus.

35. The method of claim 14, wherein said condition is an amyloid disease.

36. The method of claim 14, wherein said condition is an inflammatory disease.

37. The method of claim 14, wherein said condition is a neurodegenerative disease.

38. The method of claim 14, wherein said condition is associated with aging.

39. The method of claim 14, wherein said condition is a congenital Golgi disease (CGD).

40. The method of claim 14, wherein said condition is associated with cell senescence.

41. The method of claim 16, wherein said contacting or administering comprises an effective amount for affecting the cell in a specific manner.

42. The method of claim 14, wherein said subject is a human subject.

43. A method of diagnosing a medical condition, the method comprising analyzing activity or expression of the GQC machinery in a subject in need thereof, wherein an aberrant activity or expression of the GQC in the subject is indicative of a medical condition.
